(19) United States
(12) Patent Application Publication
(10) Pub. No.: US 2020/0215110 A1
(43) Pub. Date: Jul. 9, 2020

(54) EX VIVO ANTIGEN-PRESENTING CELLS OR ACTIVATED CD-POSITIVE T CELLS FOR TREATMENT OF INFECTIOUS DISEASES

(71) Applicant: The General Hospital Corporation, Boston, MA (US)

(72) Inventors: Mark C. Poznansky, Newton Center, MA (US); Jeffrey A. Gelfand, Cambridge, MA (US)

(21) Appl. No.: 16/331,848

(22) PCT Filed: Sep. 8, 2017
(86) PCT No.: PCT/US2017/050639

§ 371 Date: Mar. 8, 2019

Related U.S. Application Data
(60) Provisional application No. 62/385,895, filed on Sep. 9, 2016.
(60) Provisional application No. 62/385,896, filed on Sep. 9, 2016.

(51) Int. Cl. A61K 35/17; A61K 35/15; C12N 5/0783; C07K 14/54; C07K 16/08; A61K 39/385

(52) U.S. Cl. CPC A61K 35/17; A61K 35/15; C12N 5/0638; A61K 2039/6043; C07K 16/082; A61K 39/385; C07K 2319/00; C07K 14/54

(57) ABSTRACT

This disclosure is directed to methods of preparing dendritic cells or other CD40 bearing antigen-presenting cells and methods of treating an infectious disease by using the dendritic cells or other antigen-presenting cells in combination with anti-chemorepellant agents. This disclosure is further directed to methods of preparing T cells and methods of treating an infectious disease by using activated T cells optionally in combination with anti-chemorepellant agents. The antigen presenting cells of the disclosure are activated by incubation with a pathogen or pathogen-infected cells and fusion proteins. The T cells of the disclosures are activated by incubation with activated antigen-presenting cells that were activated by incubation with a pathogen or pathogen-infected cells and a fusion protein. In particular, the fusion protein comprises an antigen-binding domain, e.g., an antibody or antibody fragment, and a stress protein domain.

### STATEMENT OF PRIORITY

This application claims the benefit of U.S. Provisional Application Ser. No. 62/385,895, filed Sep. 9, 2016, and U.S. Provisional Application Ser. No. 62/385,896, filed Sep. 9, 2016, the entire contents of each of which are incorporated by reference herein.

### STATEMENT OF GOVERNMENT SUPPORT

This invention was made with government support. The U.S. Government has certain rights in this invention.

### FIELD OF THE INVENTION

This disclosure is directed to methods of preparing dendritic cells or other CD40 bearing antigen-presenting cells and methods of treating an infectious disease by using the dendritic cells or other antigen-presenting cells in combination with anti-chemorepellant agents. This disclosure is further directed to methods of preparing T cells and methods of treating an infectious disease by using activated T cells optionally in combination with anti-chemorepellant agents. The antigen presenting cells of the disclosure are activated by incubation with a pathogen or pathogen-infected cells and fusion proteins. The T cells of the disclosures are activated by incubation with activated antigen-presenting cells that were activated by incubation with a pathogen or pathogen-infected cells and a fusion protein. In particular, the fusion protein comprises an antigen-binding domain, e.g., an antibody or antibody fragment, and a stress protein domain.

### BACKGROUND OF THE INVENTION

Conventional treatments for infectious diseases, e.g., antibiotics, are often accompanied by substantial side effects. For example, antibiotics, the common medications for infectious diseases, may cause rash, diarrhea, kidney toxicity, and nausea. In addition, misuse or overuse of antibiotics can contribute to the development of more hazardous antibiotic-resistant bacteria.

Immunotherapy is a promising treatment for cancers as well as other diseases, including infectious diseases. The immunotherapy treats disease by inducing, enhancing, augmenting, or suppressing an immune response in a patient. Using immunotherapy, the immune system can be stimulated to recognize infectious pathogens, thereby creating long-lasting therapeutic effects against infectious diseases.

Therefore, there is a need for a more effective immunotherapy to promote an immune response against infectious diseases and other diseases.

### SUMMARY OF THE INVENTION

Immunotherapy is becoming an increasingly important tool in the armamentarium against infectious diseases. However, efficacy of immunotherapy has not been robust as was hoped, due in part to reduced antigenicity of the antigens and the subsequently weakened immunological response. One potential solution is to increase a patient's own immune response to disease-associated antigens by contacting T cells with antigen-presenting cells that present pathogen-specific immunogenic antigens on the cell surface. Either the antigen-presenting cells or the resulting activated T cells may be administered to the patient.

Stress proteins are very efficient at activating antigen-presenting cells to provoke a T cell response. They have been particularly effective at eliciting cell mediated immune and humoral immune responses by this pathway.

The present invention is directed to immune treatment of a pathogen infection using a fusion protein. In one aspect, the present invention is directed to a method for preparing activated antigen-presenting cells (“APCs”) by incubating immune cells ex vivo in the presence of pathogen and a fusion protein for a time period sufficient to produce the activated APCs.

In another aspect, the present invention is directed to a method for preparing pathogen-specific T cells via ex vivo activated APCs by incubating immune cells ex vivo in the presence of pathogen sample and a fusion protein for a time period sufficient to produce the activated APCs, optionally isolating the APCs from the pathogen sample and fusion protein, then incubating the T cells and activated APCs for a time period sufficient to produce activated, pathogen-specific T cells, particularly CD3-positive T cells.

The fusion protein binds to antigens with high affinity, is highly immunogenic, exhibits MHC class I priming, provokes a T cell response, and is able to be produced in non-mammalian systems such as E. coli. The fusion protein is thus suitable for use as a highly immunogenic vaccine for the prevention or treatment of infectious, inflammatory, autoimmune, or malignant disease. The fusion protein may also act as a potential candidate presented to APCs to ultimately provoke T cell responses and increase the specificity of immunotherapy against pathogens. Non-limiting examples of fusion proteins can be found in U.S. Pat. Nos. 8,143,387 and 7,943,133; U.S. Patent Pub. No. 20110129484; and PCT Application Number PCT/US2017/021911; each of which is incorporated herein by reference in its entirety.

Some pathogen-infected cells may secrete concentrations of chemokines that are sufficient to repel immune cells from the site of a pathogen infection, thus creating a “chemorepellant wall” or “fugetactic wall” around the pathogen or infected region. The chemorepellant wall reduces the immune system's ability to target and eradicate the pathogen. For example, repulsion of pathogen antigen-specific T cells, e.g., from a pathogen (or infected cells) expressing high levels of CXCL12 or interleukin 8 (IL-8), allows the pathogens or the pathogen-infected cells to evade immune control. Anti-chemorepellant agents may inhibit the chemorepellant activity of the pathogens or the pathogen-infected cells and allow the patient's immune system to target the pathogens or the pathogen-infected cells. Anti-chemorepellant agents and the systemic delivery of anti-chemorepellant agents are known in the art (see, for example, U.S. Patent Application Publication No. 2008/0300165, incorporated herein by reference in its entirety).

Without being bound by theory, it is believed that the antigen-binding domain of the fusion protein will bind to the target (e.g., pathogen, pathogen-infected cells, or pathogen sample), and the stress protein domain will induce maturation of antigen-presenting cells (e.g., dendritic cells), resulting in a T cell response to the target. In combination, the anti-chemorepellant agent will inhibit the chemorepellant activity of the target with regard to the antigen-presenting cells and/or T cells, such that the immune cells are able to penetrate the “chemorepellant wall” and access the target. In some embodiments, the combination results in additive or synergistic effects.

In one aspect, this invention relates to methods of preparing activated APCs, which comprises incubating immune cells ex vivo in the presence of pathogens or pathogen-infected cells and a fusion protein for a time period sufficient to produce the activated APCs, wherein at least one activated APC displays an antigen derived from the pathogen. In one embodiment, the method further comprises isolating the activated APCs (e.g., dendritic cells). In one embodiment, the isolated activated APCs are free or substantially free of the pathogens (and/or the pathogen-infected cells or sample) and/or the fusion protein.

The term “substantially free” as used herein refers to nearly complete removal of the pathogens (and/or the pathogen-infected cells or sample) and/or fusion protein. In one embodiment, the activated APCs are free of the pathogens (and/or the pathogen-infected cells or sample) and/or fusion protein. In one embodiment, the activated APCs are free of the pathogens (and/or the pathogen-infected cells or sample). In one embodiment, at least about 90%, at least about 91%, at least about 92%, at least about 93%, at least about 94%, at least about 95%, at least about 96%, at least about 97%, at least about 98%, at least about 99%, or at least about 99.9% of the pathogens (and/or the pathogen-infected cells or sample) are removed from the activated APCs by isolation. In one embodiment, at least about 90%, at least about 91%, at least about 92%, at least about 93%, at least about 94%, at least about 95%, at least about 96%, at least about 97%, at least about 98%, at least about 99%, or at least about 99.9% of the fusion protein is removed from the activated APCs by isolation.

In one aspect, this invention relates to methods of preparing activated T cells, which comprises incubating T cells with activated APCs for a period of time sufficient to produce activated T cells, wherein the APCs were prepared by incubating immune cells ex vivo in the presence of pathogen sample and a fusion protein for a time period sufficient to produce the activated APCs, wherein at least one activated APC displays an antigen derived from the pathogen. In one embodiment, the method further comprises isolating the activated T cells. In one embodiment, the isolated activated T cells are free or substantially free of the activated APCs, pathogen sample, and/or the fusion protein. In one embodiment, the APCs comprise dendritic cells.

In one aspect, the fusion protein comprises an antigen-binding component and a stress protein component. While the antigen-binding component binds to the pathogen sample, the stress protein component activates the APCs. In one embodiment, the antigen-binding component of the fusion protein may be any antibody or other molecule that recognizes the pathogen (and/or the pathogen-infected cell or pathogen-associated antigen) of interest. In one embodiment, the antigen-binding component is a single chain antibody, a variable domain, or a fragment antigen-binding (“Fab”) domain of an antibody.

In one aspect, the pathogen sample (e.g., pathogen, pathogen-infected cells, or pathogen-associated antigen) is obtained from a patient.

In one aspect, the antigen-binding binding component is specific for a pathogen antigen (e.g., a pathogen-specific or -associated antigen). The pathogen antigen may be any identifiable antigen that is expressed by a pathogen of interest or a pathogen-infected cell. In one embodiment, the pathogen-specific or -associated antigens include, but are not limited to, surface polysaccharide antigens, secreted toxins or other secreted antigens, or any surface antigen produced by the pathogen or infected cells.

In one embodiment, the antigen-presenting cells include, but are not limited to, dendritic cells, B lymphocytes, and mononuclear phagocytes. The mononuclear phagocytes may include monocytes and/or macrophages. In some aspects, the APCs comprise at least one CD40. In another aspect, the APCs are dendritic cells.

In one embodiment, the stress (heat shock) protein component includes but is not limited to HSP70 and/or an immune activating fragment and/or modified sequence thereof. In another aspect, the HSP70 or the immune activating fragment and/or modified sequence thereof is from Mycobacterium tuberculosis.

In one embodiment, the method of preparing the APCs further comprises expanding the immune cells in the presence of a growth factor. In one embodiment, the method of preparing the APCs further comprises expanding the activated APCs in the presence of a growth factor. In one aspect, the growth factor is a cytokine. In another aspect, the growth factor includes, but is not limited to, Flt-3 ligand, GM-CSF, IL-4, M-CSF, IFNα, IL-1β, IL-4, IL-6, IL-13, IL-15, TNFα, or any combination thereof.

The APCs (e.g., dendritic cells) may be used to treat any infectious diseases. In some embodiments, the diseases exhibit a chemorepellant effect. Therefore, the method of preparing the APCs may further comprise contacting the isolated activated APCs with an effective amount of an anti-chemorepellant agent. Anti-chemorepellant agents may include, without limitation, molecules that inhibit expression of CXCL12 or CXCR4 or CXCR7 (e.g., antisense or siRNA molecules), molecules that bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7. In one embodiment, the anti-chemorepellant agents include but are not limited to AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, an antibody that interferes with dimerization of a receptor for a chemorepellant chemokine, or a combination thereof. In another aspect, the anti-fugetactic agent is AMD3100 or derivative thereof. AMD3100 is described in U.S. Pat. No. 5,583,131, which is incorporated by reference herein in its entirety. In one embodiment, the anti-chemorepellant agent is a CXCR7 antagonist. The CXCR7 antagonist can be but is not limited to CCX771, CCX754, or an antibody that interferes with the dimerization of CXCR7. In certain embodiments, the anti-chemorepellant agent is not an antibody. In certain embodiments, the anti-chemorepellant agent is not a heparinoid. In certain embodiments, the anti-chemorepellant agent is not a peptide. In one embodiment, the amount of anti-chemorepellant agent is sufficient to bind to at least a subset of receptors, e.g., CXCR4 and/or CXCR7, on the surface of the T cells.

In one embodiment, the APCs are allogeneic, autologous, or derived from a cell line. In another embodiment, the APCs are collected from a patient having an infectious disease.

In one aspect, the APCs or pathogen sample are immobilized on a solid support. The solid support includes but is not limited to a surface of a column, a sepharose bead, a gel, a matrix, a magnetic bead, a plastic surface, or any combination thereof.

In another embodiment, the APCs are dendritic cells differentiated or matured from dendritic cell precursors. The dendritic cell precursors may be activated, or not be activated. In another aspect, the dendritic cell precursors are free or substantially free of neutrophils, macrophages, lymphocytes, or a combination thereof. In one aspect, the dendritic cell precursors are differentiated in the presence of a growth factor. The growth factor may be a cytokine. In another aspect, the growth factor is selected from a group consisting of Flt-3 ligand, GM-CSF, IL-4, M-CSF, IFNα, IL-113, IL-4, IL-6, IL-13, IL-15 and TNFα.

The methods and compositions described herein may be used to treat any pathogen infection. In some embodiments, the pathogen infection exhibits a chemorepellant effect. In one embodiment, the chemorepellant effect is mediated by overexpression of CXCL12 or other chemorepellant chemokine by the pathogen or pathogen-infected cells. In one embodiment, the method further comprises selecting a patient having an infection that exhibits a chemorepellant effect.

Another aspect of the invention relates to a method for treating an infectious disease in a patient, which comprises administering an effective amount of ex vivo prepared activated APCs to the patient, wherein the activated APCs are prepared by incubating immune cells with a pathogen sample and a fusion protein, wherein the fusion protein comprises a target or antigen binding component and a stress protein component. In particular, the target or antigen binding component of the fusion protein may bind to a pathogen sample (e.g., pathogen, pathogen-infected cells, pathogen-associated antigen), and the heat shock protein component may activate the APCs. The target or antigen binding component may be any antibody or other molecule that recognizes the pathogen of interest. In one aspect, the target or antigen binding component is a single chain antibody, a variable domain fragment, or a Fab portion of an antibody. In another aspect, the target or antigen binding component is specific for a pathogen antigen. The antigen may be any identifiable antigen that is expressed by a pathogen of interest or by a cell infected with the pathogen.

In one embodiment, the term “pathogen” refers to a pathogen sample, including but not limited to intact pathogens, viable pathogens, non-viable pathogens, viable or non-viable cells infected by a pathogen or pathogens, pathogen-specific antigens (e.g., isolated antigen, partially isolated antigen, recombinant antigen, etc.), and/or other materials derived from pathogens or pathogen-infected cells.

In one embodiment, the pathogen comprises a virus, a bacterium, a fungus, a protozoan, or a parasite, or other microorganism.

Viral causes of infectious human diseases (and their associated diseases) that can be treated by the composition, compounds and methods described herein include, but are not limited to, Influenza A virus (including ‘swine flu’ such as the 2009 H1N1 strain); Influenza B-C virus (coryza; ‘common cold’); Human adenovirus A-C (various respiratory tract infections; pneumonia); Human Para-influenza virus (coryza; ‘common cold;’ croup); Mumps virus (epidemic parotitis); Rubeola virus (measles); Rubella virus (German measles); Human respiratory syncytial virus (RSV) (coryza; ‘common cold’); Human coronavirus (SARS virus) (SARS); Human rhinovirus A-B (coryza; ‘common cold’); parvovirus B19 (fifth disease); variola virus (smallpox); varicella-zoster virus (herpes virus) (chickenpox); Human enterovirus (coryza; ‘common cold’); Bordetella pertussis (whooping cough); Neisseria meningitidis (meningitis); Corynebacterium diphtheriae (diphtheria); Mycoplasma pneumoniae (pneumonia); Mycobacterium tuberculosis (tuberculosis); Streptococcus pyogenes/pneumoniae (strep throat, meningitis, pneumonia); and Haemophilus influenzae Type B (epiglottis, meningitis, pneumonia).

In one embodiment, the virus comprises Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebola virus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Herpes simplex virus, Herpes zoster virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mononucleosis virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, SARS virus, Semliki forest virus, Seoul virus, Simian foamy virus, Simian virus 5, Sindbis virus, Southampton virus, St. Louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus and synthetic viruses.

In another embodiment, the bacterium comprises Acetobacter aurantius, Acinetobacter baumannii, Actinomyces israelii, Agrobacterium radiobacter, Agrobacterium tumefaciens, Anaplasma, Anaplasma phagocytophilum, Azorhizobium caulinodans, Azotobacter vinelandii, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacteroides fragilis, Bacteroides gingivalis, Bacteroides melaninogenicus (aka Prevotella melaninogenica), Bartonella henselae, Bartonella quintana, Bordetella, Bordetella bronchiseptica, Bordetella pertussis, Borrelia burgdorferi, Brucella, Brucella abortus, Brucella melitensis, Brucella suis, Burkholderia, Burkholderia mallei, Burkholderia pseudomallei, Burkholderia cepacia, Calymmatobacterium granulomatis, Campylobacter, Campylobacter coli, Campylobacter fetus, Campylobacter jejuni, Campylobacter pylori, Chlamydia, Chlamydia trachomatis, Chlamydophila, Chlamydophila pneumoniae (aka Chlamydia pneumoniae), Chlamydophila psittaci (aka Chlamydia psittaci), Clostridium, Clostridium botulinum, Clostridium difficile, Clostridium perfringens (aka Clostridium welchii), Clostridium tetani, Corynebacterium, Corynebacterium diphtheria, Corynebacterium fusiforme, Coxiella burnetii, Ehrlichia chaffeensis, Enterobacter cloacae, Enterococcus, Enterococcus avium, Enterococcus durans, Enterococcus faecalis, Enterococcus faecium, Enterococcus galllinarum, Enterococcus maloratus, Escherichia coli, Francisella tularensis, Fusobacterium nucleatum, Gardnerella vaginalis, Haemophilus, Haemophilus ducreyi, Haemophilus influenza, Haemophilus parainfluenzae, Haemophilus pertussis, Haemophilus vaginalis, Helicobacter pylori, Klebsiella pneumonia, Lactobacillus, Lactobacillus acidophilus, Lactobacillus bulgaricus, Lactobacillus casei, Lactococcus lactis, Legionella pneumophila, Listeria monocytogenes, Methanobacterium extroquens, Microbacterium multiforme, Micrococcus luteus, Moraxella catarrhalis, Mycobacterium, Mycobacterium avium, Mycobacterium bovis, Mycobacterium diphtheria, Mycobacterium intracellulare, Mycobacterium leprae, Mycobacterium lepraemurium, Mycobacterium phlei, Mycobacterium smegmatis, Mycobacterium tuberculosis, Mycoplasma, Mycoplasma fermentans, Mycoplasma genitalium, Mycoplasma hominis, Mycoplasma penetrans, Mycoplasma pneumonia, Neisseria, Neisseria gonorrhoeae, Neisseria meningitides, Pasteurella, Pasteurella multocida, Pasteurella tularensis, Peptostreptococcus, Porphyromonas gingivalis, Prevotella melaninogenica(aka Bacteroides melaninogenicus), Pseudomonas aeruginosa, Rhizobium radiobacter, Rickettsia, Rickettsia prowazekii, Rickettsia psittaci, Rickettsia Quintana, Rickettsia rickettsii, Rickettsia trachomae, Rochalimaea, Rochalimaea henselae, Rochalimaea Quintana, Rothia dentocariosa, Salmonella, Salmonella enteritidis, Salmonella typhi, Salmonella typhimurium, Serratia marcescens, Shigella dysenteriae, Staphylococcus, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oxalis, Streptococcus pneumonia, Streptococcus pyogenes, Streptococcus rattus, Streptococcus salivarius, Streptococcus sanguis, Streptococcus sobrinus, Treponema, Treponema pallidum, Treponema denticola, Vibrio, Vibrio cholera, Vibrio comma, Vibrio parahaemolyticus, Vibrio vulnificus, Wolbachia, Yersinia, Yersinia enterocolitica, Yersinia pestis, and Yersinia pseudotuberculosis.

In a further embodiment, the fungus comprises Acremonium; Absidia (e.g., Absidia corymbifera, etc.); Aspergillus (e.g., Aspergillus clavatus, Aspergillus flavus, Aspergillus fumigatus, Aspergillus nidulans, Aspergillus niger, Aspergillus terreus, Aspergillus verslcolor, etc.); Blastomyces (e.g., Blastomyces dermatitidis, etc.); Candida (e.g., Candida albicans, Candida glabrata, Candida guilliermondii, Candida kefyr, Candida krusei, Candida parapsilosis, Candida stellatoidea, Candida tropicalis, Candida utilis, etc.); Cladosporium (e.g., Cladosporivm trichoides, etc.); Coccidioides (e.g., Coccidioides immitis, etc.); Cryptococcus (e.g., Cryptococcus neoformans, etc.); Cunninghamella (e.g., Cunninghamella elegans, etc.); Dermatophyte; Exophiala (e.g., Exophiala dermatitidis, Exophiala spinifera, etc.); Epidermophyton (e.g., Epidermophyton floccosum, etc.); Fonsecaea (e.g., Fonsecaea pedrosoi, etc.); Fusarium (e.g., Fusarium solani, etc.); Geotrichu (e.g., Geotrichum candiddum, etc.); Histoplasma (e.g., Histoplasma capsulatum var. capsulatum, etc.); Malassezia (e.g., Malassezia furfur, etc.); Microsporum (e.g., Microsporum canis, Microsporum gypseum, etc.); Mucor; Paracoccidioides (e.g., Paracoccidioides brasiliensis, etc.); Penicillium (e.g., Penicillium arneffei, etc.); Phialophora; Pneumocystis (e.g., Pneumocystis carinii, etc.); Pseudallescheria (e.g., Pseudallescheria boydii, etc.); Rhizopus (e.g., Rhizopus microsporus var. rhizopodiformis, Rhizopus oryzae, etc.); Saccharomyces (e.g., Saccharomyces cerevisiae, etc.); Scopulariopsis; Sporothrix (e.g., Sporothrix schenckii, etc.); Trichophyton (e.g., Trichophyton mentagrophytes, Trichophyton rubru, etc.); or Trichosporon (e.g., Trichosporon asahii, Trichosporon cutaneum, etc.).

In another embodiment, this invention relates to a method for treating a disease caused by a pathogen in a patient, which comprises administering an effective amount of ex vivo prepared activated APCs to the patient, wherein the activated APCs are prepared as described herein.

In one embodiment, the non-limiting examples of the immune cells include, but are not limited to, dendritic cells, B lymphocytes, mononuclear phagocytes, and/or a combination thereof. The mononuclear phagocytes may be monocytes or macrophages. In another aspect, the immune cells express at least a CD40 receptor. In another aspect, the immune cells are dendritic cells.

The methods described herein may be used to treat a pathogen infection or an infectious disease. Preferably, the pathogen exhibits a chemorepellant effect. In one embodiment, the chemorepellant effect is mediated by overexpression of CXCL12 (e.g., at a concentration of 100 nM or greater) or other chemorepellant chemokine.

In one embodiment, the method further comprises selecting a patient infected by a pathogen that exhibits a chemorepellant effect. In another embodiment, the invention comprises administering to the patient an effective amount of an anti-chemorepellant agent. The anti-chemorepellant agent and the APCs, including dendritic cells, may be administered separately, simultaneously, and/or sequentially. In another aspect, the anti-chemorepellant agent is administered after the administration of the APCs. In another aspect, the anti-chemorepellant agent is administered before and/or during the administration of the APCs. In one aspect, the anti-chemorepellant agent is administered via injection. In one embodiment, the anti-chemorepellant agent is administered directly or proximal to the infection caused by the pathogen. In one embodiment, the anti-chemorepellant agent is administered systemically.

In one embodiment, the APCs have an anti-chemorepellant agent bound thereto via one or more cell surface receptors. The non-limiting examples of the anti-chemorepellant agents include but are not limited to AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, an antibody that interferes with dimerization of a receptor for a chemorepellant chemokine, and/or a combination thereof. In one aspect, the anti-chemorepellant agent is AMD3100. In one embodiment, the cell surface receptors comprise CXCR4 and/or CXCR7.

In one embodiment, the stress (heat shock) protein component may be HSP70 or an immune activating fragment and/or modified sequence thereof. The HSP70 or the immune activating fragment and/or modified sequence thereof may be from Mycobacterium tuberculosis. In another aspect, the target or antigen binding component comprises a single chain antibody, a variable domain, or a Fab domain.

In another embodiment, the method of treating an infectious disease in a patient further comprise expanding the immune cells, e.g., dendritic cells, in the presence of a growth factor. In one aspect, the growth factor is a cytokine. In another aspect, the non-limiting examples of the growth factors include Flt-3 ligand, GM-CSF, IL-4, M-CSF, IFNα, IL-1β, IL-4, IL-6, IL-13, IL-15, TNFα, and a combination thereof.

In one embodiment, the pathogen or pathogen-infected cell is obtained from the patient. In one embodiment, the pathogen is obtained from a source other than a patient, e.g., a pathogen culture, infected cell culture, recombinant portion of the pathogen (e.g., an antigen produced by the pathogen), or any other source.

In one embodiment, the APCs (e.g., dendritic cells) are allogeneic, autologous, or derived from a cell line. In one aspect, the APCs (e.g., dendritic cells) are collected from a patient having an infectious disease.

In another embodiment, the immune cells, APCs (e.g., dendritic cells), or the pathogen sample are immobilized on a solid support. In one aspect, the solid support comprises a column, a sepharose bead, a gel, a matrix, a magnetic bead, or a plastic surface.

In one embodiment, the dendritic cells are differentiated or matured from dendritic cell precursors. In another aspect, the dendritic cell precursors are non-activated or activated. In one aspect, the dendritic cell precursors are free or substantially free of neutrophils, macrophages, lymphocytes, or a combination thereof.

In one aspect, this disclosure relates to a method for preparing activated T cells, the method comprising:

a) providing activated antigen-presenting cells that were activated in the presence of a pathogen sample and a fusion protein ex vivo for a time period sufficient to produce activated antigen-presenting cells, wherein at least one activated antigen-presenting cell displays an antigen derived from the pathogen sample;

b) contacting the activated antigen-presenting cells with T cells for a period of time sufficient to activate the T cells; and

c) isolating the activated T cells.

In one embodiment, the isolated activated T cells are free or substantially free of the dendritic cells, pathogen sample and the fusion protein.

In one embodiment, the activated T cells target the pathogen and/or pathogen-infected cells. In one embodiment, the activated T cells target the pathogen and/or pathogen-infected cells in vivo when administered to a patient.

In one embodiment, the fusion protein comprises an antigen binding component and a stress protein component, wherein said antigen binding component binds to the pathogen sample and said stress protein component activates antigen-presenting cells.

In one embodiment, the heat shock protein component comprises HSP70 or an immune activating fragment and/or modified sequence thereof. In one embodiment, the HSP70 or the immune activating fragment and/or modified sequence thereof is from Mycobacterium tuberculosis. In one embodiment, the antigen binding component is an antibody, a single chain antibody, a variable domain, or a Fab domain.

In one embodiment, the APCs were expanded in the presence of a growth factor. In one embodiment, the growth factor is a cytokine. In one embodiment, the growth factor is selected from the group consisting of Flt-3 ligand, GM-CSF, IL-4, M-CSF, IFNα, IL-1β, IL-4, IL-6, IL-13, IL-15 and TNFα or any combination thereof.

In one embodiment, the pathogen sample is obtained from a patient. In one embodiment, the pathogen sample is derived from a patient to be treated as described herein. In one embodiment, the pathogen sample is derived from a different patient. In one embodiment, the pathogen sample comprises a cultured pathogen, secreted antigen, infected cells, infected cell line, etc.

In one embodiment, the pathogen sample my include, without limitation, intact pathogens, viable pathogens, non-viable pathogens, viable or non-viable cells infected by a pathogen or pathogens, pathogen-specific antigens (e.g., isolated antigen, partially isolated antigen, recombinant antigen, etc.), and/or other materials derived from pathogens or pathogen-infected cells.

Anti-chemorepellant agents may include, without limitation, molecules that inhibit expression of CXCL12 or CXCR4 or CXCR7 (e.g., antisense or siRNA molecules), molecules that bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7. In one embodiment, the inhibitor of CXCL12 signaling is a CXCR4 antagonist. In one embodiment, the isolated activated T cells are contacted with an effective amount of an anti-chemorepellant agent. In one embodiment, the anti-chemorepellant agent is selected from the group consisting of AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, such as CXCL12, and an antibody that interferes with dimerization of a receptor for a chemorepellant chemokine, such as CXCR4 or CXCR7. In one embodiment, the anti-chemorepellant agent is AMD3100. AMD3100 is described in U.S. Pat. No. 5,583,131, which is incorporated by reference herein in its entirety. In one embodiment, the anti-chemorepellant agent is a CXCR7 antagonist. The CXCR7 antagonist can be but is not limited to CCX771, CCX754, or an antibody that interferes with the dimerization of CXCR7. In certain embodiments, the anti-chemorepellant agent is not an antibody. In certain embodiments, the anti-chemorepellant agent is not a heparinoid. In certain embodiments, the anti-chemorepellant agent is not a peptide. In one embodiment, the amount of anti-chemorepellant agent is sufficient to bind to at least a subset of receptors, e.g., CXCR4 and/or CXCR7, on the surface of the T cells.

In one embodiment, the APCs are allogeneic, autologous, or derived from a cell line. In one embodiment, the T cells are allogeneic, autologous, or derived from a cell line. In one embodiment, the APCs and/or the T cells are isolated from a patient having an infectious disease. In some embodiments, the APCs and T cells are derived from the same source, e.g., the same patient.

In one embodiment, either the APCs or the pathogen sample are immobilized on a solid support. In one embodiment, the activated APCs are isolated by isolating the solid support from the pathogen sample and fusion protein.

In one embodiment, the T cells are immobilized on a solid support. In one embodiment, the activated T cells are isolated by isolating the solid support from the APCs.

In one embodiment, the solid support comprises a surface of a column, a sepharose bead, a gel, a matrix, a magnetic bead, or a plastic surface.

In one embodiment, this disclosure relates to a method for treating a pathogen infection in a patient, the method comprising administering an effective amount of activated T cells to the patient, wherein the activated T cells were prepared by:
providing activated APCs prepared by incubating APCs with a pathogen sample and a fusion protein, wherein the fusion protein comprises an antigen binding component and a stress protein component, wherein said antigen component binds to the pathogen sample and said stress protein component activates APCs; and
contacting the activated APCs with T cells for a period of time sufficient to activate the T cells.

In one embodiment, provided herein is a method for treating a pathogen infection in a patient, the method comprising:

a) providing activated APCs that were activated by incubating APCs in the presence of a pathogen sample and a fusion protein ex vivo for a time period sufficient to produce activated APCs, wherein at least one activated APC displays an antigen derived from the a pathogen sample;

b) contacting the activated APCs with T cells for a period of time sufficient to activate the T cells;

c) administering an effective amount of the activated T cells to the patient.

In one embodiment, the method further comprises administering to the patient an effective amount of an anti-chemorepellant agent. In one embodiment, the activated T cells have an anti-chemorepellant agent bound thereto via one or more cell surface receptors. In one embodiment, the one or more cell surface receptors comprise CXCR4 or CXCR7.

In one embodiment, the method further comprises isolating the activated T cells prior to administration to the patient. In one embodiment, the isolated activated T cells are free or substantially free of the activated APCs, pathogen sample and/or the fusion protein. In one embodiment, the fusion protein comprises an antigen binding component and a stress (e.g., heat shock) protein component, wherein said antigen binding component binds to the pathogen sample and said stress protein component activates APCs. In one embodiment, the T cells target the pathogen or infected cells, thereby treating the infection.

In one embodiment, the anti-chemorepellant agent is one described above, e.g., AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, or an antibody that interferes with dimerization of a receptor for a chemorepellant chemokine. In one embodiment, the anti-chemorepellant agent is an AMD3100 derivative. In one embodiment, the anti-chemorepellant agent is AMD3100.

In another embodiment, the dendritic cells are differentiated or matured from dendritic cell precursors. The dendritic cell precursors may be activated, or not be activated. In another aspect, the dendritic cell precursors are free or substantially free of neutrophils, macrophages, lymphocytes, or a combination thereof. In such an aspect, the term “substantially free” indicates that neutrophils, macrophages, and/or lymphocytes comprise less than about 10%, e.g., less than about 5%, e.g., less than about 2%, e.g., less than about 1% of the total cells in a dendritic cell precursor composition.

In one aspect, the dendritic cell precursors are differentiated in the presence of a growth factor. The growth factor may be a cytokine. In another aspect, the growth factor is selected from a group consisting of Flt-3 ligand, GM-CSF, IL-4, M-CSF, IFNα, IL-1β, IL-4, IL-6, IL-13, IL-15 and TNFα.

The methods described herein may be used to treat any pathogen infection. In one embodiment, the pathogenic infection exhibits a chemorepellant effect. In one embodiment, the chemorepellant effect is mediated by overexpression of CXCL12 or other chemorepellant chemokine by the pathogen or pathogen-infected cells. In one embodiment, the method further comprises selecting a patient having an infection that exhibits a chemorepellant effect.

In one embodiment, the pathogen comprises a virus, a bacterium, a fungus, a protozoan, or a parasite, or other microorganism, e.g., any of the pathogens listed above.

In another embodiment, this invention relates to a method for treating a disease caused by a pathogen in a patient, which comprises administering an effective amount of ex vivo prepared activated T cells to the patient, wherein the activated T cells are prepared by incubating T cells with activated APCs. In one embodiment, the activated APCs are/were prepared by incubating immune cells with pathogens and a fusion protein, wherein the fusion protein comprises a target or antigen binding component and a heat shock protein component, wherein the target binding component binds to the pathogen and/or cells infected by the pathogen, and the stress protein component activates the APCs. In one embodiment, the pathogen is a virus, a protozoan, a bacterium, a parasite, or a fungus.

In one embodiment, examples of the immune cells include, but are not limited to, dendritic cells, B lymphocytes, mononuclear phagocytes, and/or a combination thereof. The mononuclear phagocytes may be monocytes or macrophages. In another aspect, the immune cells bear at least a CD40 receptor. In another aspect, the immune cells are dendritic cells.

The methods described herein may be used to treat a pathogen or an infectious disease. In one embodiment, the pathogen exhibits a chemorepellant effect. In one embodiment, the chemorepellant effect is mediated by overexpression of CXCL12 or other chemorepellant chemokine. In one embodiment, the method further comprises selecting a patient having an infectious disease which exhibits a chemorepellant effect. In one embodiment, the chemorepellant effect is mediated by CXCL12.

In another embodiment, the invention further comprises administering to the patient an effective amount of an anti-chemorepellant agent, an anti-pathogen agent, and/or a fusion protein. The anti-chemorepellant agent, anti-pathogen agent, and/or fusion protein and the T cells, may be administered separately, simultaneously, and/or sequentially. In another aspect, the anti-chemorepellant agent, anti-pathogen agent, and/or fusion protein is administered after the administration of the T cells. In another aspect, the anti-chemorepellant agent, anti-pathogen agent, and/or fusion protein is administered before and/or during the administration of the T cells.

In one aspect, the anti-chemorepellant agent, anti-pathogen agent, and/or fusion protein is administered via injection. In one embodiment, the anti-chemorepellant agent, anti-pathogen agent, and/or fusion protein is administered directly or proximal to the infection caused by the pathogen. In one embodiment, the anti-chemorepellant agent, anti-pathogen agent, and/or fusion protein is administered systemically.

In one embodiment, the activated T cells have an anti-chemorepellant agent bound thereto via one or more cell surface receptors. In one embodiment, the one or more cell surface receptors comprise CXCR4 or CXCR7.

In one embodiment, the anti-chemorepellant agent is one described above, e.g., AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, an antibody that interferes with dimerization of a receptor for a chemorepellant chemokine, and/or a combination thereof. In one aspect, the anti-chemorepellant agent is AMD3100.

In one embodiment, the stress (heat shock) protein component may be HSP70 or an immune activating fragment and/or modified sequence thereof. The HSP70 or the immune activating fragment and/or modified sequence thereof may be from Mycobacterium tuberculosis. In another aspect, the target or antigen binding component comprises a single chain antibody, a variable domain, or a Fab domain.

In one embodiment, the pathogen sample is obtained from the patient. In one embodiment, the pathogen sample is obtained from a source other than a patient, e.g., a different infected patient, a pathogen culture, infected cell culture, recombinant portion of the pathogen (e.g., an antigen produced by the pathogen), or any other source.

In one embodiment, the APCs (e.g., dendritic cells) are allogeneic, autologous, or derived from a cell line. In one aspect, the APCs (e.g., dendritic cells) are collected from a patient having an infectious disease.

In one embodiment, the T cells are allogeneic, autologous, or derived from a cell line. In one aspect, the T cells are collected from a patient having an infectious disease. In one embodiment, the T cells and APCs are derived from the same source (e.g., the same patient or donor).

In another embodiment, the immune cells, APCs (e.g., dendritic cells), or the T cells are immobilized on a solid support. In one aspect, the solid support comprises a column, a sepharose bead, a gel, a matrix, a magnetic bead, or a plastic surface.

In one embodiment, the dendritic cells are differentiated or matured from dendritic cell precursors. In another aspect, the dendritic cell precursors are non-activated or activated. In one aspect, the dendritic cell precursors are free or substantially free of neutrophils, macrophages, lymphocytes, or a combination thereof.

In one embodiment, this invention relates to a composition comprising immune cells and an effective amount of a fusion protein, said fusion protein comprising an antigen-binding component and a stress protein component.

In one embodiment, provided herein is a pharmaceutical composition comprising ex vivo activated APCs and an anti-chemorepellant agent. In one embodiment, the APCs were activated by a method as described herein.

In one embodiment, the activated APCs have an anti-chemorepellant agent bound thereto via one or more cell surface receptors. In one embodiment, the one or more cell surface receptors comprise CXCR4 and/or CXCR7.

In one embodiment, the composition further comprises a pharmaceutically acceptable excipient. In one embodiment, the composition further comprises an effective amount of a fusion protein as described herein.

In one embodiment, the composition comprises activated APCs and a fusion protein. In one embodiment, the composition comprises an effective amount of the fusion protein to elicit an anti-pathogen immune response in an infected patient.

In one embodiment, this invention relates to a composition comprising activated APCs and T cells. In one embodiment, the composition further comprises a fusion protein. In one embodiment, the composition comprises a complex, wherein the complex includes an activated APC and a T cell, and optionally a fusion protein.

In one embodiment, this invention relates to a pharmaceutical composition comprising ex vivo activated T cells and an effective amount of a fusion protein, said fusion protein comprising a pathogen binding component and a stress protein component. In one embodiment, the composition further comprises an anti-chemorepellant agent. In one embodiment, the composition further comprises an anti-pathogen agent.

In one embodiment, provided herein is a pharmaceutical composition comprising ex vivo activated T cells and an anti-chemorepellant agent. In one embodiment, the composition further comprises an anti-pathogen agent.

In one embodiment, provided herein is a pharmaceutical composition comprising ex vivo activated T cells and an anti-pathogen agent.

In one embodiment, the T cells were activated by a method as described herein.

In one embodiment, the activated T cells have an anti-chemorepellant agent bound thereto via one or more cell surface receptors. In one embodiment, the one or more cell surface receptors comprise CXCR4 and/or CXCR7.

In one embodiment, the composition further comprises a pharmaceutically acceptable excipient.

### DETAILED DESCRIPTION

After reading this description, it will become apparent to one skilled in the art how to implement the invention in various alternative embodiments and alternative applications. However, not all embodiments of the present invention are described herein. It will be understood that the embodiments presented here are presented by way of an example only, and not limitation. As such, this detailed description of various alternative embodiments should not be construed to limit the scope or breadth of the present invention as set forth below.

Before the present invention is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

### Definitions

Unless defined otherwise, all technical and scientific terms used herein have the same meaning as commonly understood by one of ordinary skill in the art to which this invention belongs.

In this specification and in the claims that follow, reference will be made to a number of terms that shall be defined to have the following meanings:

The terminology used herein is for the purpose of describing particular embodiments only and is not intended to be limiting the invention. As used herein, the singular forms “a,” “an” and “the” are intended to include the plural forms as well, unless the context clearly indicates otherwise.

All numerical designations, e.g., pH, temperature, time, concentration, amounts, and molecular weight, including ranges, are approximations which are varied (+) or (−) by 10%, 1%, or 0.1%, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

The term “pathogen” or “pathogen sample” includes, but is not limited to, intact pathogens, viable pathogens, non-viable pathogens, viable or non-viable cells infected by a pathogen or pathogens, pathogen-specific antigens (e.g., isolated antigen, partially isolated antigen, recombinant antigen, etc.), and/or other materials derived from pathogens or pathogen-infected cells.

A “pathogen” or “human pathogen” may be any bacteria, fungi, protozoa, parasites, and viruses, as well as other microorganisms that cause human diseases.

The term “antigen-presenting cell” or “APC” refers to an immune cell that is capable of presenting an antigen to the immune system (e.g., T cells) including dendritic cells, mononuclear phagocytes (e.g., monocytes and macrophages), B lymphocytes, and the like, from a mammal (e.g., human or mouse). The antigen may be presented on the surfaces of the antigen-presenting cells.

The term “dendritic cell” refers to one type of antigen-presenting cell capable of activating T cells and/or stimulating the differentiation of B cells.

The term “CD40 receptor” refers to a costimulatory protein, peptide, or polypeptide, or a nucleic acid encoding the peptide, polypeptide or protein. The CD40 receptor may be present in immune cells, including but not limited to APCs.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of,” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In one embodiment, the patient, subject, or individual is a mammal. In some embodiments, the mammal is a mouse, a rat, a guinea pig, a non-human primate, a dog, a cat, or a domesticated animal (e.g., horse, cow, pig, goat, sheep). In some embodiments, the patient, subject or individual is a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disease or disorder; (iii) slowing progression of the disease or disorder; and/or (iv) inhibiting, relieving, or slowing progression of one or more symptoms of the disease or disorder. For example, treatment of an infectious disease includes, but is not limited to, reduction in size of infection, elimination of the infection, remission of the infection, reduction or elimination of at least one symptom of the infection, and the like.

The term “administering” or “administration” of an agent to a subject includes any route of introducing or delivering to a subject a compound to perform its intended function. Administration can be carried out by any suitable route, including orally, intranasally, parenterally (intravenously, intramuscularly, intraperitoneally, or subcutaneously), or topically. Administration includes self-administration and the administration by another.

“Pharmaceutically acceptable composition” refers to a composition that is suitable for administration to a mammal, particularly, a human.

It is also to be appreciated that the various modes of treatment or prevention of medical diseases and conditions as described are intended to mean “substantial,” which includes total but also less than total treatment or prevention, and wherein some biologically or medically relevant result is achieved.

The phrase “concurrently administering” refers to administration of at least two agents to a patient over a period of time. As used herein, the word “concurrently” means sufficiently close in time to produce a combined effect (that is, concurrently can be simultaneously, or it can be two or more events occurring within a short time period before or after each other). Concurrent administration includes, without limitation, separate, sequential, and simultaneous administration.

The term “separate” administration refers to an administration of at least two active ingredients at the same time or substantially the same time by different routes or in different compositions.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients.

The term “simultaneous” administration refers to the administration of at least two active ingredients by the same route and at the same time or at substantially the same time.

The term “therapeutic” as used herein means a treatment. A therapeutic effect is obtained by suppression, remission, or eradication of a disease state.

The term “prevent” or “preventative” as used herein means a prophylactic treatment. A preventative effect is obtained by delaying the onset of a disease state or decreasing the severity of a disease state when it occurs.

The term “therapeutically effective amount,” “prophylactically effective amount,” or “effective amount” refers to an amount of the agent that, when administered, is sufficient to cause the desired effect. For example, an effective amount of activated T cells may be an amount sufficient to treat a pathogen infection. The therapeutically effective amount of the agent may vary depending on the pathogen being treated and its severity as well as the age, weight, etc., of the patient to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

The term “kill” with respect to a pathogen or cell/cell population is directed to include any type of manipulation that will lead to the death of that pathogen or cell/cell population.

The terms “antibodies” and “antibody” as used herein include polyclonal, monoclonal, single chain, chimeric, humanized and human antibodies, prepared according to conventional methodology. Antibody may also refer to a portion of an antibody, preferably a portion which binds an antigen (e.g., single chain antibody, Fab or similar fragment, etc.).

The term “CXCR4/CXCL12 antagonist” or “CXCR7/CXCL12 antagonist” refers to a compound that antagonizes CXCL12 binding to CXCR4 and/or CXCR7 or otherwise reduces the chemorepellant effect of CXCL12.

By “chemorepellant activity” or “chemorepellant effect” it is meant the ability of an agent to repel (or chemorepel) a eukaryotic cell with migratory capacity (i.e., a cell that can move away from a repellant stimulus), as well as the chemorepellant effect of a chemokine secreted by a cell, e.g., a pathogen-infected cell. Usually, the chemorepellant effect is present in an area around the cell wherein the concentration of the chemokine is sufficient to provide the chemorepellant effect. Some chemokines, including interleukin 8 and CXCL12, may exert chemorepellant activity at high concentrations (e.g., over about 100 nM), whereas lower concentrations exhibit no chemorepellant effect and may even be chemoattractant.

Accordingly, an agent with chemorepellant activity is a “chemorepellant agent.” Such activity can be detected using any of a variety of systems well known in the art (see, e.g., U.S. Pat. No. 5,514,555 and U.S. Patent Application Pub. No. 2008/0300165, each of which is incorporated by reference herein in its entirety). A preferred system for use herein is described in U.S. Pat. No. 6,448,054, which is incorporated herein by reference in its entirety.

The term “anti-chemorepellant effect” refers to the effect of the anti-chemorepellant agent to attenuate or eliminate the chemorepellant effect of the chemokine.

The term “autogeneic” or “autologous” refers to the origin of a cell, when the cell administered to an individual is derived from the individual or a genetically identical individual (i.e., an identical twin of the individual). An autogeneic cell can also be a progeny of an autogeneic cell. The term also indicates that cells of different cell types are derived from the same individuals or genetically identical individuals.

The term “allogeneic” or “allogenic” also refers to the origin of a cell, when the cell being administered to an individual is derived from an individual not genetically identical to the recipient but from the same species, including a progeny of an allogeneic cell. Cells are still called allogeneic when they are of different cell types but are derived from genetically non-identical donors, or when they are progenies of cells derived from genetically non-identical donors.

“Immune cells” as used herein are cells of hematopoietic origin that are involved in the specific recognition of antigens. Immune cells include APCs, such as dendritic cells or macrophages, B cells, T cells, etc.

The term “anti-pathogen therapy” or “anti-infectious disease therapy” as used herein refers to traditional infection treatments, including antibiotics, antiviral agents, antifungal agents, anti-parasitic agents, and antimicrobial agents, as well as immunotherapy and vaccine therapy.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (e.g., IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

A “fusion protein” or “fusion polypeptide” refers to a hybrid polypeptide which comprises polypeptide portions from at least two different polypeptides. The portions may be from proteins of the same organism, in which case the fusion protein is said to be “intraspecies,” “intragenic,” etc. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. A first polypeptide may be fused to the N-terminus, the C-terminus, or the N- and C-terminus of a second polypeptide. Furthermore, a first polypeptide may be inserted within the sequence of a second polypeptide.

The term “immunogenic” refers to the ability of a substance to elicit an immune response. An “immunogenic composition” or “immunogenic substance” is a composition or substance which elicits an immune response. An “immune response” refers to the reaction of a subject to the presence of an antigen, which may include at least one of the following: making antibodies, developing immunity, developing hypersensitivity to the antigen, and developing tolerance.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

As used herein, a “stress protein” also known as a “heat shock protein” or “HSP” is a protein that is encoded by a stress gene, and is therefore typically produced in significantly greater amounts upon the contact or exposure of the stressor to the organism. The term “stress protein” as used herein is intended to include such portions and peptides of a stress protein. A “stress gene,” also known as “heat shock gene,” as used herein, refers to a gene that is activated or otherwise detectably upregulated due to the contact or exposure of an organism (containing the gene) to a stressor, such as but not limited to heat shock, hypoxia, glucose deprivation, heavy metal salts, inhibitors of energy metabolism and electron transport, and protein denaturants, or to certain benzoquinone ansamycins. Nover, L., Heat Shock Response, CRC Press, Inc., Boca Raton, Fla. (1991). “Stress gene” also includes homologous genes within known stress gene families, such as certain genes within the HSP70 and HSP90 stress gene families, even though such homologous genes are not themselves induced by a stressor. Each of the terms stress gene and stress protein as used in the present specification may be inclusive of the other, unless the context indicates otherwise.

The term “vaccine” refers to a substance that elicits an immune response and also confers protective immunity upon a subject.

### Activated Antigen-Presenting Cells (APCs)

### Antigen-Presenting Cells (APCs)

The antigen-presenting cells (APCs) may include dendritic cells, B lymphocytes, mononuclear phagocytes (e.g., monocytes and/or macrophages), and/or other cell types expressing the necessary MHC/co-stimulatory molecules. In one aspect, the APCs comprise a costimulatory protein, e.g., CD40, which is important for the activation of the APCs. In one embodiment, the APCs may be presented with antigens by pulsing, in which the APCs are exposed to antigenic proteins or polypeptides. The proteins or peptides may be recombinant or natural products. In another embodiment, APCs are exposed to the nucleic acids encoding the proteins or peptides in the presence of transfection agents known in the art, including but not limited to cationic lipids.

Transfected or pulsed APCs can subsequently be administered to the host via an intravenous, subcutaneous, intranasal, intramuscular, or intraperitoneal route of delivery. Protein/peptide antigens can also be delivered in vivo with adjuvant via the intravenous, subcutaneous, intranasal, intramuscular or intraperitoneal route of delivery.

According to the invention, foster antigen-presenting cells may also be used as a substitute for the antigen-presenting cells. The foster antigen-presenting cells lack antigen processing activity, whereby they express MHC molecules free of bound peptides. In one aspect, foster APCs are derived from the human cell line 174X CEM.T2, referred to as T2, which contains a mutation in its antigen processing pathway that restricts the association of endogenous peptides with cell surface MHC class I molecules. Zweerink et al., J. Immunol. 150:1763-1771 (1993). Transduction of T2 cells with specific recombinant MHC alleles allows for redirection of the MHC restriction profile. Libraries tailored to the recombinant allele will be preferentially presented by them because the anchor residues will prevent efficient binding to the endogenous allele. High level expression of MHC molecules makes the APCs more visible to the CTLs. Expressing the MHC allele of interest in T2 cells using a powerful transcriptional promoter (e.g., the CMV promoter) results in a more reactive APC.

In one embodiment, the APCs are modified to express or over-express co-stimulatory molecules. Co-stimulatory molecules include, but are not limited to, CD40, MHC class I and/or class II, B7-1 (CD80), B7-2 (CD86), ICAM-1, interleukin 1 (IL-1), and LFA-3. See, e.g., Hodge et al. J Natl Cancer Inst (2000) 92 (15): 1228-1239, which is incorporated herein by reference in its entirety.

### Dendritic Cells

As one type of antigen-presenting cells, dendritic cells are effective in presenting antigens and initiating immune responses, particularly the immune responses associated with T cells. Dendritic cells are present in skin, nose, lung, intestines, blood, and many other tissues and organs. The immunogenicity of the dendritic cells is partly due to their ability to process antigen materials and present them on the cell surface.

After capturing the antigens, the dendritic cells can present the antigen to T cells. The dendritic cells become mature with increased surface expression of class II MHC and costimulatory molecules. Santambrogio et al., PNAS, 96(26): 15056-15061 (1999). Mature dendritic cells may lose the ability to further process exogenous antigens. Maturation of the dendritic cells may also be accompanied with the loss of acidic organelles, where the antigens were processed. Kampgen et al., PNAS, 88: 3014-3018 (1991).

The efficiency of the dendritic cells may be limited by the small number of the cells in any given organ. For example, around 0.1% of the white cells exist as dendritic cells in human blood. Dendritic cells from some organs (e.g., spleen and afferent lymphatic) arise from precursors. Thus, in one aspect of the invention, dendritic cells are obtained or expanded from dendritic cell precursors. The methods of expanding or obtaining dendritic cells are described in U.S. Pat. No. 5,994,126, which is incorporated herein by reference in its entirety.

### Methods of Making Activated Antigen Presenting Cells

The activated APCs used in the methods described herein to activate T cells are activated using a fusion protein and pathogen sample.

In one embodiment, the APCs used in the methods described herein are activated by incubating an effective amount of a fusion protein with immune cells and pathogen sample under conditions so as to produce activated APCs. In one embodiment, the APCs are activated by a method which comprises incubating immune cells ex vivo in the presence of pathogen sample and a fusion protein for a time period sufficient to produce the activated APCs, wherein at least one activated APC displays an antigen derived from the pathogen sample. In one embodiment, the method further comprises isolating the activated APCs. In one embodiment, the isolated activated APCs are free or substantially free of the pathogen sample and/or the fusion protein.

The term “substantially free” as used herein refers to nearly complete removal of the pathogen sample and/or fusion protein. In one embodiment, the activated APCs are free of the pathogen sample and/or fusion protein. In one embodiment, the activated APCs are free of the pathogen sample. In one embodiment, at least about 90%, at least about 91%, at least about 92%, at least about 93%, at least about 94%, at least about 95%, at least about 96%, at least about 97%, at least about 98%, at least about 99%, or at least about 99.9% of the pathogen sample is removed from the activated APCs by isolation. In one embodiment, at least about 90%, at least about 91%, at least about 92%, at least about 93%, at least about 94%, at least about 95%, at least about 96%, at least about 97%, at least about 98%, at least about 99%, or at least about 99.9% of the fusion protein is removed from the activated APCs by isolation.

In one aspect, the fusion protein comprises an antigen-binding component and a stress protein component. While the antigen-binding component binds to the pathogen sample, the stress protein component activates the APCs. In one embodiment, the antigen-binding component of the fusion protein may be any antibody or other molecule (e.g., aptamer) that recognizes a pathogen sample (e.g., pathogen, pathogen-derived antigen, pathogen-associated antigen, infected cell, etc.) of interest. In one embodiment, the antigen-binding component is a single chain antibody, a variable domain, or a fragment antigen-binding (“Fab”) domain of an antibody.

In one aspect, the pathogen sample, e.g., pathogen, infected cells, or other pathogen-associated material, is obtained from a patient.

In one embodiment, the immune cells (APCs) include, but are not limited to, dendritic cells, B lymphocytes, and mononuclear phagocytes. The mononuclear phagocytes may include monocytes and/or macrophages. In some aspects, the immune cells comprise at least one CD40. In another aspect, the immune cells are dendritic cells.

In one embodiment, the stress (heat shock) protein component includes but is not limited to HSP70 and/or an immune activating fragment and/or modified sequence thereof. In another aspect, the HSP70 or the immune activating fragment and/or modified sequence thereof is from Mycobacterium tuberculosis.

In one embodiment, the method of preparing the APCs further comprises expanding the immune cells in the presence of a growth factor. In one embodiment, the method of preparing the APCs further comprises expanding the activated APCs in the presence of a growth factor. In one aspect, the growth factor is a cytokine. In another aspect, the growth factor includes, but is not limited to, Flt-3 ligand, GM-CSF, IL-4, M-CSF, IFNα, IL-1β, IL-4, IL-6, IL-13, IL-15, TNFα, or any combination thereof.

In one embodiment, the APCs are allogeneic, autologous, or derived from a cell line. In another embodiment, the APCs are collected from a patient having a pathogen infection. In one aspect, either the APCs or the pathogen sample are immobilized on a solid support. The solid support includes but is not limited to a surface of a column, a sepharose bead, a gel, a matrix, a magnetic bead, a plastic surface, or any combination thereof.

In another embodiment, the dendritic cells are differentiated or matured from dendritic cell precursors. The dendritic cell precursors may be activated, or not be activated. In another aspect, the dendritic cell precursors are free or substantially free of neutrophils, macrophages, lymphocytes, or a combination thereof. In such an aspect, the term “substantially free” indicates that neutrophils, macrophages, and/or lymphocytes comprise less than about 10%, e.g., less than about 5%, e.g., less than about 2%, e.g., less than about 1% of the total cells in a dendritic cell precursor composition.

In one aspect, the dendritic cell precursors are differentiated in the presence of a growth factor. The growth factor may be a cytokine. In another aspect, the growth factor is selected from a group consisting of Flt-3 ligand, GM-CSF, IL-4, M-CSF, IFNα, IL-13, IL-4, IL-6, IL-13, IL-15 and TNFα.

In one embodiment, the APCs are modified to express or over-express co-stimulatory molecules. Co-stimulatory molecules include, but are not limited to, CD40, MHC class I and/or class II, B7-1 (CD80), B7-2 (CD86), ICAM-1, interleukin 1 (IL-1), and LFA-3. See, e.g., Hodge et al. J Natl Cancer Inst (2000) 92 (15): 1228-1239, which is incorporated herein by reference in its entirety.

### Anti-Chemorepellant Agents

Many pathogens or pathogen-infected cells have chemorepellant effects, e.g., on immune cells, due to chemokines secreted by the pathogens or the pathogen-infected cells. High concentrations of the chemokines secreted by the pathogens or the pathogen-infected cells can have chemorepellant effects on cells, whereas lower concentrations do not have such effects or even result in chemoattraction. For example, T cells are repelled by CXCL12 (SDF-1) by a concentration-dependent and CXCR4 receptor-mediated mechanism.

The anti-chemorepellant agent may be any such agent known in the art, for example an anti-chemorepellant agent as described in U.S. Patent Application Publication No. 2008/0300165, which is hereby incorporated by reference in its entirety.

Anti-chemorepellant agents include any agents that specifically inhibit chemokine and/or chemokine receptor dimerization, thereby blocking the chemorepellant response to a chemorepellant agent. Certain chemokines, including IL-8 and CXCL12 can also serve as chemorepellants at high concentrations (e.g., above 100 nM) where many of the chemokines exist as a dimer. Dimerization of the chemokines elicits a differential response in cells, causing dimerization of chemokine receptors, an activity which is interpreted as a chemorepellant signal. Blocking the chemorepellant effect of high concentrations of chemokines secreted by pathogen-infected cells can be accomplished, for example, by anti-chemorepellant agents which inhibit chemokine dimer formation or chemokine receptor dimer formation. For example, antibodies that target and block chemokine receptor dimerization, e.g., by interfering with the dimerization domains or ligand binding, can be anti-chemorepellant agents. Anti-chemorepellant agents that act via other mechanisms of action, e.g., that reduce the amount of chemorepellant cytokine secreted by the cells, inhibit dimerization, and/or inhibit binding of the chemokine to a target receptor, are also encompassed by the present invention. Where desired, this effect can be achieved without inhibiting the chemotactic action of monomeric chemokine.

In other embodiments, the anti-chemorepellant agent is a CXCR4 antagonist, CXCR7 antagonist, CXCR3 antagonist, CXCR4/CXCL12 antagonist, CXCR7/CXCL12 antagonist, or selective PKC inhibitor. Anti-chemorepellant agents may include, without limitation, molecules that inhibit expression of CXCL12 or CXCR4 or CXCR7 (e.g., antisense or siRNA molecules), molecules that bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7.

The CXCR4 antagonist can be but is not limited to AMD3100 (plerixafor) or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, or TN14003, derivatives thereof, or an antibody that interferes with the dimerization of CXCR4. Additional CXCR4 antagonists are described, for example, in U.S. Patent Pub. No. 2014/0219952 and Debnath et al. Theranostics, 2013; 3(1): 47-75, each of which is incorporated herein by reference in its entirety, and include TG-0054 (burixafor), AMD3465, NIBR1816, AMD070, and derivatives thereof.

The CXCR3 antagonist can be but is not limited to TAK-779, AK602, or SCH-351125, or an antibody that interferes with the dimerization of CXCR3.

The CXCR4/CXCL12 antagonist can be but is not limited to tannic acid, NSC 651016, or an antibody that interferes with the dimerization of CXCR4 and/or CXCL12.

The CXCR7/CXCL12 antagonist can be but is not limited to CCX771, CCX754, or an antibody that interferes with the dimerization of CXCR7 and/or CXCL12.

The selective PKC inhibitor can be but is not limited to thalidomide or GF 109230X.

In one embodiment, the anti-chemorepellant agent is AMD3100 (plerixafor). AMD3100 is described in U.S. Pat. No. 5,583,131, which is incorporated by reference herein in its entirety.

In one embodiment, the anti-chemorepellant agent is an AMD3100 derivative. AMD3100 derivatives include, but are not limited to, those found in U.S. Pat. Nos. 7,935,692 and 5,583,131 (USRE42152), each of which is incorporated herein by reference in its entirety.

In certain embodiments, the anti-chemorepellant agent is not an antibody. In certain embodiments, the anti-chemorepellant agent is not a heparinoid. In certain embodiments, the anti-chemorepellant agent is not a peptide.

In one embodiment, the anti-chemorepellant agent is coupled with a molecule that allows targeting of a pathogen or a pathogen-infected cell. In one embodiment, the anti-chemorepellant agent is coupled with (e.g., bound to) an antibody specific for the pathogen or a pathogen-infected cell to be targeted. In one embodiment, the anti-chemorepellant agent coupled to the molecule that allows targeting of the pathogen or a pathogen-infected cell is administered systemically.

In one embodiment, the anti-chemorepellant agent is administered in combination with an additional compound that enhances the anti-chemorepellant activity of the agent. In one embodiment, the additional compound is granulocyte colony stimulating factor (“G-CSF”). In one embodiment, G-CSF is not administered.

### Fusion Protein

This disclosure relates to fusion proteins comprising a stress protein component and a target or antigen binding component, and methods of using the same. In particular, this disclosure relates to treating a patient having a disease, e.g., an infectious disease that can be recognized by a fusion protein as described herein. Preferably, the disease expresses a chemorepellant activity such that immune cells are inhibited in the vicinity of the diseased cells, pathogen, or tumor cells.

Examples and methods of making fusion proteins contemplated in the present invention are described in U.S. Pat. Nos. 8,143,387 and 7,943,133 and PCT Application Number PCT/US2017/021911, each of which is incorporated herein by reference in its entirety.

### Stress Protein Component

The stress protein component (also referred to as the stress protein domain or heat shock component) may comprise any polypeptide sequence that activates APCs. In some embodiments, the polypeptide sequence is derived from a stress protein. However, any APC-activating polypeptide is contemplated.

Any suitable stress protein (e.g., heat shock protein) can be used in the fusion polypeptides of the present invention. For example, HSP60 and/or HSP70 can be used. Turning to stress proteins generally, cells respond to a stressor (typically heat shock treatment) by increasing the expression of a group of genes commonly referred to as stress, or heat shock, genes. Heat shock treatment involves exposure of cells or organisms to temperatures that are one to several degrees Celsius above the temperature to which the cells are adapted. In coordination with the induction of such genes, the levels of corresponding stress proteins increase in stressed cells.

In bacteria, the predominant stress proteins are proteins with molecular sizes of about 70 kDa and 60 kDa, which are commonly referred to as HSP70 and HSP60, respectively. Stress proteins appear to participate in important cellular processes such as protein synthesis, intracellular trafficking, and assembly and disassembly of protein complexes. It appears that the increased amounts of stress proteins synthesized during stress serve primarily to minimize the consequences of induced protein unfolding. Indeed, the pre-exposure of cells to mildly stressful conditions that induce the synthesis of stress proteins affords protection to the cells from the deleterious effects of a subsequent, more extreme stress.

The major stress proteins appear to be expressed in every organism and tissue type examined so far. Also, it appears that stress proteins represent the most highly conserved group of proteins identified to date. For example, when stress proteins in widely diverse organisms are compared, HSP90 and HSP70 exhibit 50% or higher identity at the amino acid level and share many similarities at non-identical positions. Similar or higher levels of homology exist between different members of a particular stress protein family within species.

The stress proteins, particularly HSP 70, HSP 60, HSP 20-30 and HSP 10, are among the major determinants recognized by the host immune system in the immune response to infection by Mycobacterium tuberculosis and Mycobacterium leprae. However, individuals, including healthy individuals with no history of mycobacterial infection or autoimmune disease, also carry T cells that recognize both bacterial and human HSP 60 epitopes. This system recognizing stress protein epitopes presumably constitutes an “early defense system” against invading organisms. The system may be maintained by frequent stimulation by bacteria and viruses.

Families of stress genes and proteins for use in the fusion polypeptides are those well known in the art and include, for example, HSP 100-200, HSP 100, HSP 90, Lon, HSP 70, HSP 60, TF55, HSP 40, FKBPs, cyclophilins, HSP 20-30, ClpP, GrpE, HSP 10, ubiquitin, calnexin, and protein disulfide isomerases. In certain embodiments, the stress protein is HSP 70 or HSP 60. In certain embodiments, the stress protein is a fragment of Hsp70 or Hsp60 and/or a modified sequence of Hsp70 or Hsp60. As use herein, a “modified sequence” of a stress protein such as Hsp70 is a sequence comprising one or more additions, deletion, or substitutions that retains at least 50% of at least one of the biological activity of the stress protein, e.g., the ability to stimulate antigen presenting cells, e.g., at least 50%, 60%, 70%, 80%, or more of the biological activity. In some embodiments, the modified sequence has an enhanced biological activity compared to the wild-type sequence. In some embodiments, the modified sequence is one disclosed in PCT Application No. PCT/US2017/021911, incorporated by reference herein in its entirety.

The examples of HSP 100-200 include Grp170 (for glucose-regulated protein). HSP 100 examples include mammalian HSP 110, yeast HSP 104, ClpA, ClpB, ClpC, ClpX and ClpY. HSP 90 examples include HtpG in E. coli, HSP 83 and Hsc83 in yeast, and HSP 90alpha, HSP 90beta and Grp94 in humans. HSP 70 examples include HSP 72 and Hsc73 from mammalian cells, DnaK from bacteria, particularly mycobacteria such as Mycobacterium leprae, Mycobacterium tuberculosis, and Mycobacterium bovis (such as Bacille-Calmette Guerin, referred to herein as Hsp71), DnaK from Escherichia coli, yeast, and other prokaryotes, and BiP and Grp78. HSP 60 examples include HSP 65 from mycobacteria. Bacterial HSP 60 is also commonly known as GroEL, such as the GroEL from E. coli. TF55 examples include Tep1, TRiC and thermosome. HSP 40 examples include DnaJ from prokaryotes such as E. coli and mycobacteria and HSJ1, HDJ1 and HSP 40. FKBPs examples include FKBP12, FKBP13, FKBP25, and FKBP59, Fpr1 and Nep1. Cyclophilin examples include cyclophilinsA, Band C. HSP 10 examples include GroES and Cpn10.

In particular embodiments, the stress proteins of the present invention are obtained from enterobacteria, mycobacteria (particularly M. leprae, M. tuberculosis, M. vaccae, M. smegmatis and M. bovis), E. coli, yeast, Drosophila, vertebrates, avians, chickens, mammals, rats, mice, primates, or humans.

In one embodiment, the stress protein comprises Mycobacterium tuberculosis-derived heat shock protein 70 (MtbHsp70). MtbHsp70 is well characterized and functions as a potent immune-activating adjuvant. It stimulates monocytes and dendritic cells (DCs) to produce CC-chemokines, which attract antigen processing and presenting macrophages, DCs, and effector T and B cells.

A fusion polypeptide may comprise an amino acid sequence that is at least about 80%, 85%, 90%, 95%, 98%, or 99% identical to a stress protein described herein. Preferably, the amino acid sequence retains the ability to activate APCs.

A fusion polypeptide may comprise an amino acid sequence which is a fragment and/or modification of the stress protein as described herein. Preferably, the amino acid sequence retains the ability to activate APCs.

### Target Binding Component

The target binding component of the fusion protein may be any molecule that specifically binds an antigen associated with the disease to be treated. In certain embodiments, the target binding component is an antibody or a fragment thereof. The terms “antigen-binding,” “target binding,” and “pathogen-binding” are used interchangeably herein.

In one aspect, the target binding component is a single chain antibody. In one aspect, the target binding component is a variable domain fragment. In one aspect, the target binding component is a Fab portion of an antibody.

In one aspect, the target binding component is specific for a pathogen antigen (e.g., a pathogen-specific antigen or a pathogen-associated antigen), and may be referred to as a pathogen binding component. The pathogen antigen may be any identifiable antigen or epitope that is expressed by a pathogen of interest or the pathogen-infected cell. In one embodiment, the antigen is a surface antigen (e.g., expressed on the surface of the pathogen and/or infected cells). In one embodiment, the pathogen antigens include, but are not limited to, surface polysaccharide antigens.

In one aspect, the target binding component is specific for an antigen associated with or produced by a pathogen or a pathogen-infected cell. Suitable pathogens and antigens can be found, for example and without limitation, in U.S. Pat. No. 8,143,387, which is incorporated herein by reference in its entirety.

In one embodiment, the target binding component (e.g., antibody) is specific for an epitope associated with an infectious disease. In one embodiment, the target binding component (e.g., antibody) is specific for an epitope associated with a microbial infection, such as a bacterial infection. In one embodiment, the infectious disease is caused by or related to a pathogen selected from the group consisting of bacteria, fungi, viruses, protozoa, parasites, or other microorganisms. In one embodiment, the target binding component (e.g., antibody) is specific for an epitope associated with the pathogen. In one embodiment, the target binding component (e.g., antibody) is specific for an epitope associated with a toxin produced by the pathogen. In one embodiment, the target binding component (e.g., antibody) targets one or more symptoms of the infectious disease.

In one embodiment, the antibodies include, but are not limited to, Palivizumab, Actoxumab, Bezlotoxumab, CR6261, Diridavumab, Edobacomab, Efungumab, Exbivirumab, Felvizumab, Firivumab, Foravirumab, Ibalizumab, Libivirumab, Motavizumab, Obiltoxaximab, Pagibaximab, Panobacumab, Pritoxaximab, PRO 140, VRC01LS, Rafivirumab, Raxibacumab, Regavirumab, Setoxaximab, Sevirumab, Suvizumab, Tefibazumab, Tosatoxumab, Tuvirumab, Urtoxazumab, ado-trastuzumab emtansine, alemtuzumab, bevacizumab, cetuximab, denosumab, dinutuximab, ipilimumab, nivolumab, obinutuzumab, ofatumumab, panitumumab, pembrolizumab, pertuzumab, rituximab, and trastuzumab.

Table 1 depicts a non-limiting list of antibodies approved or in trials for infectious disease targets.

TABLE 1
Antibodies
Antibodies

| Biologic | Type | Treatment(s) | Target(s) |
|---|---|---|---|
| Palivizumab | Humanized | Respiratory syncytial virus | RSVF |
| | | | protein |
| Actoxumab | Human | Clostridium difficile colitis | Exotoxin |
| | | | TcdA |
| Bezlotoxumab | Human | Clostridium difficile infection | Exotoxin |
| | | | TcdB |

Table 2 lists a fusion protein for an infectious disease target.

TABLE 2
Fusion protein
Fusion Proteins

| | | | |
|---|---|---|---|
| Biologic | Description | Treatment(s) | Target(s) |

| | | |
|---|---|---|
| N/A | Toll-like receptor 4 | Bacterial sepsis |
| | with IgG1 Fc | |

Table 3 lists other antibodies for infectious disease uses.

TABLE 3
Other antibodies for infectious disease uses
Antibodies

| Antibody | Type | Proposed Treatment/Target |
|---|---|---|
| Bezlotoxumab | human | Clostridium difficile colitis |
| CR6261 | human | infectious disease, influenza A |
| Diridavumab | human | influenza A |
| Edobacomab | mouse | sepsis caused by Gram-negative bacteria |
| Efungumab | human | invasive Candida infection |
| Exbivirumab | human | hepatitis B |
| Felvizumab | humanized | respiratory syncytial virus infection |
| Firivumab | human | influenza |
| Foravirumab | human | rabies |
| Ibalizumab | humanized | HIV infection |
| Libivirumab | human | hepatitis B |
| Motavizumab | humanized | respiratory syncytial virus |
| Obiltoxaximab | chimeric | Bacillus anthracis spores |
| Pagibaximab | chimeric | sepsis (Staphylococcus) |
| Panobacumab | human | Pseudomonas aeruginosa infection |
| Pritoxaximab | chimeric | Anti-Shiga toxin 1 B subunit |
| PRO 140 | humanized | HIV infection |
| VRC01LS | humanized | HIV |
| Rafivirumab | human | rabies |
| Raxibacumab | human | anthrax (prophylaxis and treatment) |
| Regavirumab | human | cytomegalovirus infection |
| Setoxaximab | chimeric | E. coli |
| Sevirumab | human | cytomegalovirus infection |
| Suvizumab | humanized | viral infections |
| Tefibazumab | humanized | Staphylococcus aureus infection |
| Tosatoxumab | human | Anti-S. aureus alpha-toxin |
| Tuvirumab | human | chronic hepatitis B |
| Urtoxazumab | humanized | diarrhea caused by E. coli |

### Bacterial Toxins

Many bacteria have the ability to produce toxins, which is an underlying mechanism by which bacterial pathogens produce disease. Bacterial toxins include lipopolysaccharides, which are associated with the cell wall of Gram-negative bacteria, and proteins, which are released from bacterial cells and may act at tissue sites removed from the site of bacterial growth. Endotoxins are cell-associated toxins and exotoxins are the extracellular diffusible toxins.

In one embodiment, the target binding component (e.g., antibody) is specific for a bacterial toxin or an epitope associated with the bacterial toxin, including an endotoxin or an exotoxin.

Endotoxins include, but are not limited to, the lipopolysaccharides (LPS) or lipooligosaccharides (LOS) that are located in the outer membrane of Gram-negative bacteria. Endotoxins can be released from growing bacteria or from cells that are lysed as a result of effective host defense mechanisms or by the activities of certain antibiotics.

Endotoxins are part of the outer membrane of the cell wall of Gram-negative bacteria. Endotoxin is invariably associated with Gram-negative bacteria whether the organisms are pathogenic or not. Although the term “endotoxin” is occasionally used to refer to any cell-associated bacterial toxin, in bacteriology it is properly reserved to refer to the lipopolysaccharide complex associated with the outer membrane of Gram-negative pathogens such as Escherichia coli, Salmonella, Shigella, Pseudomonas, Neisseria, Haemophilus influenzae, Bordetella pertussis and Vibrio cholerae.

Enterotoxins are bacterial exotoxins that have an action upon the intestinal mucosa. They may be produced within the intestine by pathogenic bacteria. Bacterial enterotoxins are potent mucosal immunogens that activate both mucosal and systemic immune responses, and thus are the cause of various diseases, which include food poisoning, common diarrhea, colitis, chronic inflammation and dysentery.

### Activated T Cells

In one aspect, this disclosure relates to methods of preparing activated, pathogen-targeting T cells from APCs that were activated using a fusion protein as described herein.

In one embodiment, this invention relates to methods of preparing activated T cells, which comprises incubating T cells with activated antigen-presenting cells (APCs) for a period of time sufficient to produce activated T cells, wherein the APCs were prepared by incubating immune cells ex vivo in the presence of a pathogen sample and a fusion protein for a time period sufficient to produce the activated APCs, wherein at least one activated APC displays an antigen derived from the pathogen sample. In one embodiment, the method further comprises isolating the activated T cells, wherein the isolated activated T cells are free or substantially free of the activated APCs, pathogen sample and/or the fusion protein. In one embodiment, the APCs comprise dendritic cells.

The term “substantially free” as used with respect to the activated T cells refers to nearly complete removal of the APCs and/or pathogen sample and/or fusion protein. In one embodiment, the activated T cells are free of the APCs and/or pathogen sample and/or fusion protein. In one embodiment, the activated T cells are free of the pathogen sample. In one embodiment, at least about 90%, at least about 91%, at least about 92%, at least about 93%, at least about 94%, at least about 95%, at least about 96%, at least about 97%, at least about 98%, at least about 99%, or at least about 99.9% of the pathogen sample is removed from the activated T cells by isolation. In one embodiment, at least about 90%, at least about 91%, at least about 92%, at least about 93%, at least about 94%, at least about 95%, at least about 96%, at least about 97%, at least about 98%, at least about 99%, or at least about 99.9% of the fusion protein is removed from the activated T cells by isolation. In one embodiment, at least about 90%, at least about 91%, at least about 92%, at least about 93%, at least about 94%, at least about 95%, at least about 96%, at least about 97%, at least about 98%, at least about 99%, or at least about 99.9% of the APCs are removed from the activated T cells by isolation.

In one aspect, the fusion protein comprises an antigen-binding component and a stress (e.g., heat shock) protein component. While the antigen-binding component binds to the pathogen sample, the stress protein component activates the APCs. In one embodiment, the antigen-binding component of the fusion protein may be any antibody or other molecule that recognizes a pathogen, pathogen-infected cell, or other pathogen sample (e.g., pathogen-associated antigen) of interest. In one embodiment, the antigen-binding component is a single chain antibody, a variable domain, or a fragment antigen-binding (“Fab”) domain of an antibody.

In one aspect, the pathogen sample is derived from a patient infected with the pathogen.

In one embodiment, the antigen-presenting cells include, but are not limited to, dendritic cells, B lymphocytes, and mononuclear phagocytes. The mononuclear phagocytes may include monocytes and/or macrophages. In some aspects, the APCs comprise at least one CD40. In another aspect, the APCs are dendritic cells. In some aspects, the APCs express one or more costimulatory proteins.

In one embodiment, the stress (heat shock) protein component includes but is not limited to HSP70 and/or an immune activating fragment and/or modified sequence thereof. In another aspect, the HSP70 or the immune activating fragment and/or modified sequence thereof is from Mycobacterium tuberculosis.

In one embodiment, this disclosure relates to a method for preparing activated T cells, the method comprising:

a) providing activated antigen-presenting cells that were activated in the presence of a pathogen sample and a fusion protein ex vivo for a time period sufficient to produce activated antigen-presenting cells, wherein at least one activated antigen-presenting cell displays an antigen derived from the pathogen sample; and

b) contacting the activated antigen-presenting cells with T cells for a period of time sufficient to activate the T cells.

In one embodiment, the method further comprises isolating the activated T cells. In one embodiment, the isolated activated T cells are free or substantially free of the APCs, pathogen sample, and the fusion protein.

In one embodiment, the fusion protein comprises an antigen binding component and a stress protein component, wherein said antigen binding component binds to the pathogen sample and said stress protein component causes activation of antigen-presenting cells. In one embodiment, the stress protein component comprises HSP70 or an immune activating fragment and/or modified sequence thereof. In one embodiment, the HSP70 or the immune activating fragment and/or modified sequence thereof is from Mycobacterium tuberculosis. In one embodiment, the antigen binding component is a single chain antibody, a variable domain, or a Fab domain.

In one embodiment, the APCs were expanded in the presence of a growth factor. In one embodiment, the growth factor is a cytokine. In one embodiment, the growth factor is selected from the group consisting of Flt-3 ligand, GM-CSF, IL-4, M-CSF, IFNα, IL-1β, IL-4, IL-6, IL-13, IL-15 and TNFα.

In one embodiment, the APCs are selected from the group consisting of dendritic cells, B lymphocytes, mononuclear phagocytes, and any combination thereof. In one embodiment, the APCs are dendritic cells.

In one embodiment, the dendritic cells are expanded in presence of a growth factor. In one embodiment, the growth factor is a cytokine. In one embodiment, the growth factor is selected from the group consisting of Flt-3 ligand, GM-CSF, IL-4, M-CSF, IFNα, IL-1β, IL-4, IL-6, IL-13, IL-15 and TNFα.

In one embodiment, the pathogen sample is obtained from a patient. In one embodiment, the pathogen sample is obtained from the patient to be treated with the T cells.

In one embodiment, the isolated activated T cells are contacted with an effective amount of an anti-chemorepellant agent. In one embodiment, the anti-chemorepellant agent is any of the agents disclosed previously, e.g., selected from the group consisting of AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, and an antibody that interferes with dimerization of a receptor for a chemorepellant chemokine. In one embodiment, the anti-chemorepellant agent is AMD3100. In one embodiment, the amount of anti-chemorepellant agent is sufficient to bind to at least a subset of receptors, e.g., CXCR4 and/or CXCR7, on the surface of the T cells.

In one embodiment, the APCs and/or T cells are allogeneic, autologous, or derived from a cell line. In one embodiment, the APCs and/or the T cells are isolated from a patient infected with the pathogen.

In one embodiment, the APCs are immobilized on a solid support. In one embodiment, the activated T cells are isolated by removing the solid support from the T cells.

In one embodiment, the T cells are immobilized on a solid support. In one embodiment, the activated T cells are isolated by isolating the solid support from the APCs.

In one embodiment, the solid support comprises a surface of a column, a sepharose bead, a gel, a matrix, a magnetic bead, or a plastic surface.

### Methods of Treatment

The activated APCs as described herein can be used to treat a pathogen infection in a patient. The disease may be mediated by a pathogen, such as a virus, a bacterium, a fungus, a parasite, a protozoan, or a microorganism. In another embodiment, the disease is an infectious disease.

Another aspect of the invention relates to a method for treating an infectious disease in a patient, which comprises administering an effective amount of ex vivo prepared APCs to the patient, wherein the APCs are prepared by incubating immune cells with a pathogen (and/or a pathogen-infected cell) and a fusion protein, wherein the fusion protein comprises a target or antigen binding component and a stress protein component. In particular, the target or antigen binding component of the fusion protein may bind to pathogens (and/or the pathogen-infected cells), and the stress protein component may activate the APCs. The target or antigen binding component may be any antibody or other molecule that recognizes the pathogen of interest. In one aspect, the target or antigen binding component is a single chain antibody, a variable domain fragment, or a Fab portion of an antibody. In another aspect, the target or antigen binding component is specific for a pathogen antigen. The antigen may be any identifiable antigen that is expressed by a pathogen of interest or by a cell infected with the pathogen.

In one embodiment, the pathogen comprises a virus, a bacterium, a fungus, a protozoan, or a parasite, or other microorganism.

In another embodiment, this invention relates to a method for treating a disease caused by a pathogen in a patient, which comprises administering an effective amount of ex vivo prepared activated APCs to the patient, wherein the activated APCs are prepared by incubating immune cells with pathogens and a fusion protein, wherein the fusion protein comprises a target or antigen binding component and a stress protein component, wherein the target binding component binds to the pathogen and/or cells infected by the pathogen, and the stress protein component activates the APCs. In one embodiment, the pathogen is a virus, a bacterium, a parasite, or a fungus.

In one embodiment, examples of the immune cells include, but are not limited to, dendritic cells, B lymphocytes, mononuclear phagocytes, and/or a combination thereof. The mononuclear phagocytes may be monocytes or macrophages. In another aspect, the immune cells bear at least a CD40 receptor. In another aspect, the immune cells are dendritic cells.

The methods described herein may be used to treat a pathogen or an infectious disease. In one embodiment, the pathogen exhibits a chemorepellant effect. In one embodiment, the chemorepellant effect is mediated by overexpression of CXCL12 or other chemorepellant chemokine, e.g., expresses an amount of CXCL12 in the microenvironment sufficient to have a chemorepellant effect. In one embodiment, the method comprises selecting a patient having an infectious disease which exhibits a chemorepellant effect, e.g., is determined to express or over-express CXCL12, e.g., a concentration of about 100 nM or higher, e.g., about 100, 150, 200, 250, 300, 350, 400, 450, or 500 nM.

In one embodiment, the method further comprises selecting a patient infected by a pathogen that exhibits a chemorepellant effect. In another embodiment, the invention further comprises administering to the patient an effective amount of an anti-chemorepellant agent. The anti-chemorepellant agent and the APCs, including dendritic cells, may be administered separately, simultaneously, and/or sequentially. In another aspect, the anti-chemorepellant agent is administered after the administration of the APCs, including dendritic cells. In another aspect, the anti-chemorepellant agent is administered before and/or during the administration of the APCs (e.g., dendritic cells).

In one aspect, the anti-chemorepellant agent and/or APCs is administered via injection. In one embodiment, the anti-chemorepellant agent and/or APCs is administered directly or proximal to the infection caused by the pathogen. In one embodiment, the anti-chemorepellant agent and/or APCs is administered systemically.

In one embodiment, the APCs have an anti-chemorepellant agent bound thereto via one or more cell surface receptors. In one embodiment, the anti-chemorepellant agent is any of the agents disclosed previously, e.g., including but are not limited to AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, an antibody that interferes with dimerization of a receptor for a chemorepellant chemokine, and/or a combination thereof. In one aspect, the anti-chemorepellant agent is AMD3100.

In one embodiment, the heat shock protein component may be HSP70 or an immune activating fragment and/or modified sequence thereof. The HSP70 or the immune activating fragment and/or modified sequence thereof may be from Mycobacterium tuberculosis.

In another aspect, the target or antigen binding component comprises a single chain antibody, a variable domain, or a Fab domain.

In another embodiment, the method of treating an infectious disease in a patient further comprises expanding the immune cells, e.g., dendritic cells, in the presence of a growth factor. In one aspect, the growth factor is a cytokine. In another aspect, the non-limiting examples of the growth factors include Flt-3 ligand, GM-CSF, IL-4, M-CSF, IFNα, IL-1β, IL-4, IL-6, IL-13, IL-15, TNFα, and a combination thereof.

In one embodiment, the pathogen or pathogen-infected cell is obtained from the patient to be treated. In one embodiment, the pathogen is obtained from a source other than the patient, e.g., a different infected patient, a pathogen culture, infected cell culture, recombinant portion of the pathogen (e.g., an antigen produced by the pathogen), or any other source.

In one embodiment, the APCs (e.g., dendritic cells) are allogeneic, autologous, or derived from a cell line. In one aspect, the APCs (e.g., dendritic cells) are collected from a patient having an infectious disease. In one aspect, the APCs (e.g., dendritic cells) are collected from the patient to be treated.

In another embodiment, the immune cells, APCs (e.g., dendritic cells), or the pathogen sample are immobilized on a solid support. In one aspect, the solid support comprises a column, a sepharose bead, a gel, a matrix, a magnetic bead, or a plastic surface. In one embodiment, the APCs are separated from the pathogen sample and/or fusion protein by removing/separating the solid support. Where the APCs are immobilized on a solid support, the APCs are preferably removed from the solid support prior to administration to a patient.

In one embodiment, the dendritic cells are differentiated or matured from dendritic cell precursors. In another aspect, the dendritic cell precursors are non-activated or activated. In one aspect, the dendritic cell precursors are free or substantially free of neutrophils, macrophages, lymphocytes, or a combination thereof. In such an aspect, the term “substantially free” indicates that neutrophils, macrophages, and/or lymphocytes comprise less than about 10%, e.g., less than about 5%, e.g., less than about 2%, e.g., less than about 1% of the total cells in a dendritic cell precursor composition.

In one embodiment, the anti-chemorepellant agent and the APCs (e.g., dendritic cells) are administered separately. In one embodiment, the anti-chemorepellant agent and the APCs (e.g., dendritic cells) are administered simultaneously. In one embodiment, the anti-chemorepellant agent and the APCs (e.g., dendritic cells) are administered sequentially.

In one embodiment, the anti-chemorepellant agent is administered prior to administration of the APCs (e.g., dendritic cells). In one embodiment, the anti-chemorepellant agent is administered after administration of the APCs (e.g., dendritic cells). In one embodiment, the anti-chemorepellant agent is administered before, during and/or after administration of the APCs (e.g., dendritic cells).

In some embodiments, the anti-chemorepellant agent is administered between one minute and 24 hours prior to administration of the APCs (e.g., dendritic cells). In some embodiments, the anti-chemorepellant agent is administered 1, 2, 3, 4, 5, 6, or 7 days prior to administration of the APCs (e.g., dendritic cells).

In some embodiments, the anti-chemorepellant agent is administered for a period of time sufficient to reduce or attenuate the chemorepellant effect of the pathogen, e.g., such that the anti-chemorepellant agent has an anti-chemorepellant effect; the APCs (e.g., dendritic cells) can then be administered for a period of time during which the chemorepellant effect of the pathogen is reduced or attenuated.

In some embodiments, the anti-chemorepellant agent is administered between one minute and 24 hours after administration of the APCs (e.g., dendritic cells). In some embodiments, the anti-chemorepellant agent is administered 1, 2, 3, 4, 5, 6, or 7 days after administration of the APCs (e.g., dendritic cells).

In one embodiment, the anti-chemorepellant agent and/or the APCs (e.g., dendritic cells) is administered intravenously, subcutaneously, orally, or intraperitoneally. In one embodiment, the anti-chemorepellant agent is administered proximal to (e.g., near or within the same body cavity as) the pathogen or the pathogen-infected cell or region/tissue. In one embodiment, the anti-chemorepellant agent is administered directly into the pathogen, the pathogen-infected cell, the pathogen-infected region or tissue, or into a blood vessel feeding the pathogen-infected cell, region, or tissue. In one embodiment, the anti-chemorepellant agent is administered systemically. In a further embodiment, the anti-chemorepellant agent is administered by microcatheter, an implanted device, or an implanted dosage form.

In one embodiment, the anti-chemorepellant agent is administered in a continuous manner for a defined period. In another embodiment, the anti-chemorepellant agent is administered in a pulsatile manner. For example, the anti-chemorepellant agent may be administered intermittently over a period of time.

In one embodiment, the method further comprises administering tot the patient an effective amount of an anti-pathogen agent that targets the pathogen of interest or the pathogen-infected cells. The anti-pathogen therapy may be any therapy that targets a pathogen, including but not limited to an antimicrobial agent, an antibiotic, an anti-fungal agent, an anti-parasitic agent, an anti-protozoa agent, an anti-viral agent, and the like.

In one embodiment, the method further comprises administering to the patient an effective amount of a fusion protein as described herein having an antigen binding component that binds to an antigen associated with the pathogen infection. The fusion protein may be administered before, at approximately the same time as, or after administration of the activated APCs and/or anti-chemorepellant agent. Dosages and routes of administration of the fusion protein are provided, for example in U.S. Pat. Nos. 8,143,387 and 7,943,133; U.S. Patent Pub. No. 20110129484; and U.S. Provisional Application No. 62/306,168; each of which is incorporated herein by reference in its entirety.

The activated T cells as described herein can be used to treat an infection by a pathogen in a patient that expresses an antigen recognized by the activated T cells. The disease may be mediated by a pathogen, such as a virus, a bacterium, a fungus, a parasite, a protozoan, or a microorganism. In another embodiment, the disease is an infectious disease.

The activated T cells as described herein can be used in combination with an anti-chemorepellant agent to treat a disease which is associated with a chemorepellant effect.

In one embodiment is provided a method for treating a pathogen infection in a patient, the method comprising administering an effective amount of activated T cells to the patient, wherein the activated T cells were prepared by:

incubating antigen-resenting cells (APCs) with a pathogen sample and a fusion protein for a period of time sufficient to activate the APCs; and
contacting the activated APCs with T cells for a period of time sufficient to activate the T cells.

In one embodiment, the fusion protein comprises a stress protein component. In one embodiment, the stress protein component comprises HSP70 or an immune activating fragment and/or modified sequence thereof. In one embodiment, the HSP70 or the immune activating fragment and/or modified sequence thereof is from Mycobacterium tuberculosis.

In one embodiment, the fusion protein comprises an antigen binding component. In one embodiment, the antigen binding component comprises a single chain antibody, a variable domain, or a Fab domain.

In one embodiment, the pathogen sample is obtained from the patient. In one embodiment, the pathogen sample is derived from a different patient. In one embodiment, the pathogen sample comprises a cultured pathogen, secreted antigen, infected cells, infected cell line, etc.

In one embodiment, the APCs are allogeneic, autologous, or derived from a cell line. In one embodiment, the APCs are collected from the patient.

In one embodiment, the T cells are allogeneic, autologous, or derived from a cell line. In one embodiment, the T cells are collected from the patient. In one embodiment, the APCs and the T cells are derived from the same patient.

In one embodiment, either the activated APCs or the T cells are immobilized on a solid support. In one embodiment, the solid support comprises a surface of a column, a sepharose bead, a gel, a matrix, a magnetic bead, or a plastic surface.

In one embodiment, the APCs are dendritic cells, and the dendritic cells are differentiated or matured from dendritic cell precursors.

In one embodiment is provided a method for treating a pathogen infection in a patient, the method comprising:

a) providing activated APCs that were activated by incubating APCs in the presence of a pathogen sample and a fusion protein ex vivo for a time period sufficient to produce activated APCs, wherein at least one activated APC displays an antigen derived from the pathogen sample;

b) contacting the activated APCs with T cells for a period of time sufficient to activate the T cells;

c) optionally isolating the activated T cells; and

d) administering an effective amount of the activated T cells to the patient.

In one embodiment, the isolated activated T cells are free or substantially free of the activated APCs, pathogen sample, and the fusion protein.

In one embodiment, the activated T cells have an anti-chemorepellant agent bound thereto via one or more cell surface receptors.

In one embodiment, the method further comprises administering to the patient an effective amount of an anti-chemorepellant agent. In one embodiment, the anti-chemorepellant agent is any of the agents disclosed previously, e.g., including but are not limited to AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, and an antibody that interferes with dimerization of a receptor for a chemorepellant chemokine. In a preferred embodiment, the anti-chemorepellant agent is AMD3100.

In one embodiment, the anti-chemorepellant agent and the activated T cells are administered separately, e.g., by the same or different routes. In one embodiment, the anti-chemorepellant agent and the activated T cells are administered simultaneously, in the same or different compositions, e.g., by the same or different routes. In one embodiment, the anti-chemorepellant agent and the activated T cells are administered sequentially, e.g., by the same or different routes.

In one embodiment, the anti-chemorepellant agent is administered prior to administration of the activated T cells. In one embodiment, the anti-chemorepellant agent is administered after administration of the activated T cells. In one embodiment, the anti-chemorepellant agent is administered before, during and/or after administration of the activated T cells.

In some embodiments, the anti-chemorepellant agent is administered between one minute and 24 hours prior to administration of the activated T cells. In some embodiments, the anti-chemorepellant agent is administered 1, 2, 3, 4, 5, 6, or 7 days prior to administration of the activated T cells.

In some embodiments, the anti-chemorepellant agent is administered for a period of time sufficient to reduce or attenuate the chemorepellant effect of the pathogen, e.g., such that the anti-chemorepellant agent has an anti-chemorepellant effect; the activated T cells can then be administered for a period of time during which the chemorepellant effect of the pathogen is reduced or attenuated.

In some embodiments, the anti-chemorepellant agent is administered between one minute and 24 hours after administration of the activated T cells. In some embodiments, the anti-chemorepellant agent is administered 1, 2, 3, 4, 5, 6, or 7 days after administration of the activated T cells.

In one embodiment, the anti-chemorepellant agent and/or the activated T cells is administered intravenously, subcutaneously, orally, or intraperitoneally. In one embodiment, the anti-chemorepellant agent is administered proximal to (e.g., near or within the same body cavity as) the pathogen or the pathogen-infected cell or region/tissue. In one embodiment, the anti-chemorepellant agent is administered directly into the pathogen, the pathogen-infected cell, the pathogen-infected region or tissue, or into a blood vessel feeding the pathogen-infected cell, region, or tissue. In one embodiment, the anti-chemorepellant agent is administered systemically. In a further embodiment, the anti-chemorepellant agent is administered by microcatheter, an implanted device, or an implanted dosage form.

In one embodiment, the anti-chemorepellant agent is administered in a continuous manner for a defined period. In another embodiment, the anti-chemorepellant agent is administered in a pulsatile manner. For example, the anti-chemorepellant agent may be administered intermittently over a period of time.

In one embodiment, the method further comprises administering to the patient an effective amount of an anti-pathogen agent that targets the pathogen of interest or the pathogen-infected cells. The anti-pathogen therapy may be any therapy that targets a pathogen, including but not limited to an antimicrobial agent, an antibiotic, an anti-fungal agent, an anti-parasitic agent, an anti-protozoa agent, an anti-viral agent, and the like.

In one aspect, the pathogen can be virus, bacteria, protozoa, parasite, or fungus. In another embodiment, the infectious diseases can be caused by virus, bacteria, protozoa, parasite, or a fungus.

In one embodiment, the method further comprises administering to the patient an effective amount of a fusion protein as described herein having an antigen binding component that binds to an antigen associated with the pathogen infection. The fusion protein may be administered before, at approximately the same time as, or after administration of the activated T cells and/or anti-chemorepellant agent. Dosages and routes of administration of the fusion protein are provided, for example in U.S. Pat. Nos. 8,143,387 and 7,943,133; U.S. Patent Pub. No. 20110129484; and U.S. Provisional Application No. 62/306,169; each of which is incorporated herein by reference in its entirety.

In one embodiment, the infectious disease can be caused by bacteria, viruses, fungi, parasites, or other microorganism, e.g., as listed above. The non-limiting list of infectious diseases and their sources that can be treated by the composition, compounds, and the methods described herein are listed in the Table 4.

TABLE 4
A non-limiting list of infectious diseases.

| Disease | Source of Disease |
|---|---|
| Acinetobacter infections | Acinetobacter baumannii |
| Actinomycosis | Actinomyces israelii, Actinomyces gerencseriae and |
| | Propionibacterium propionicus |
| African sleeping sickness (African | Trypanosoma brucei |
| trypanosomiasis) | |
| AIDS (Acquired immunodeficiency | HIV (Human immunodeficiency virus) |
| syndrome) | |
| Amebiasis | Entamoeba histolytica |
| Anaplasmosis | Anaplasma species |
| Angiostrongyliasis | Angiostrongylus |
| Anisakiasis | Anisakis |
| Anthrax | Bacillus anthracis |
| Arcanobacterium haemolyticum infection | Arcanobacterium haemolyticum |
| Argentine hemorrhagic fever | Junin virus |
| Ascariasis | Ascaris lumbricoides |
| Aspergillosis | Aspergillus species |
| Astrovirus infection | Astroviridae family |
| Babesiosis | Babesia species |
| Bacillus cereus infection | Bacillus cereus |
| Bacterial pneumonia | multiple bacteria |
| Bacterial vaginosis | List of bacterial vaginosis microbiota |
| Bacteroides infection | Bacteroides species |
| Balantidiasis | Balantidium coli |
| Bartonellosis | Bartonella |
| Baylisascaris infection | Baylisascaris species |
| BK virus infection | BK virus |
| Black piedra | Piedraia hortae |
| Blastocystosis | Blastocystis species |
| Blastomycosis | Blastomyces dermatitidis |
| Bolivian hemorrhagic fever | Machupo virus |
| Botulism (and Infant botulism) | Clostridium botulinum; Note: Botulism is not an |
| | infection by Clostridium botulinum but caused by the |
| | intake of botulinum toxin. |
| Brazilian hemorrhagic fever | Sabia virus |
| Brucellosis | Brucella species |
| Bubonic plague | the bacterial family Enterobacteriaceae |
| Burkholderia infection | usually Burkholderia cepacia and other Burkholderia |
| | species |
| Buruli ulcer | Mycobacterium ulcerans |
| Calicivirus infection (Norovirus and | Caliciviridae family |
| Sapovirus) | |
| Campylobacteriosis | Campylobacter species |
| Candidiasis (Moniliasis; Thrush) | usually Candida albicans and other Candida species |
| Capillariasis | Intestinal disease by Capillaria philippinensis, hepatic |
| | disease by Capillaria hepatica and pulmonary disease by |
| | Capillaria aerophila |
| Carrion's disease | Bartonella bacilliformis |
| Cat-scratch disease | Bartonella henselae |
| Cellulitis | usually Group A Streptococcus and Staphylococcus |
| Chagas Disease (American | Trypanosoma cruzi |
| trypanosomiasis) | |
| Chancroid | Haemophilus ducreyi |
| Chickenpox | Varicella zoster virus (VZV) |
| Chikungunya | Alphavirus |
| Chlamydia | Chlamydia trachomatis |
| Chlamydophila pneumoniae infection | Chlamydophila pneumoniae |
| (Taiwan acute respiratory agent or TWAR) | |
| Cholera | Vibrio cholerae |
| Chromoblastomycosis | usually Fonsecaea pedrosoi |
| Chytridiomycosis | Batrachochytrium dendrabatidis |
| Clonorchiasis | Clonorchis sinensis |
| Clostridium difficile colitis | Clostridium difficile |
| Coccidioidomycosis | Coccidioides immitis and Coccidioides posadasii |
| Colorado tick fever (CTF) | Colorado tick fever virus (CTFV) |
| Common cold (Acute viral | usually rhinoviruses and coronaviruses |
| rhinopharyngitis; Acute coryza) | |
| Creutzfeldt-Jakob disease (CJD) | PRNP |
| Crimean-Congo hemorrhagic fever (CCHF) | Crimean-Congo hemorrhagic fever virus |
| Cryptococcosis | Cryptococcus neoformans |
| Cryptosporidiosis | Cryptosporidium species |
| Cutaneous larva migrans (CLM) | usually Ancylostoma braziliense; multiple other parasites |
| Cyclosporiasis | Cyclospora cayetanensis |
| Cysticercosis | Taenia solium |
| Cytomegalovirus infection | Cytomegalovirus |
| Dengue fever | Dengue viruses (DEN-1, DEN-2, DEN-3 and DEN-4) - |
| | Flaviviruses |
| Desmodesmus infection | Green algae Desmodesmus armatus |
| Dientamoebiasis | Dientamoeba fragilis |
| Diphtheria | Corynebacterium diphtheriae |
| Diphyllobothriasis | Diphyllobothrium |
| Dracunculiasis | Dracunculus medinensis |
| Ebola hemorrhagic fever | Ebolavirus (EBOV) |
| Echinococcosis | Echinococcus species |
| Ehrlichiosis | Ehrlichia species |
| Enterobiasis (Pinworm infection) | Enterobius vermicularis |
| Enterococcus infection | Enterococcus species |
| Enterovirus infection | Enterovirus species |
| Epidemic typhus | Rickettsia prowazekii |
| Erythema infectiosum (Fifth disease) | Parvovirus B19 |
| Exanthem subitum (Sixth disease) | Human herpesvirus 6 (HHV-6) and Human herpesvirus 7 |
| | (HHV-7) |
| Fasciolasis | Fasciola hepatica and Fasciola gigantica |
| Fasciolopsiasis | Fasciolopsis buski |
| Fatal familial insomnia (FFI) | PRNP |
| Filariasis | Filarioidea superfamily |
| Food poisoning by Clostridium perfringens | Clostridium perfringens |
| Free-living amebic infection | multiple |
| Fusobacterium infection | Fusobacterium species |
| Gas gangrene (Clostridial myonecrosis) | usually Clostridium perfringens; other Clostridium |
| | species |
| Geotrichosis | Geotrichum candidum |
| Gerstmann-Straussler-Scheinker syndrome | PRNP |
| (GSS) | |
| Giardiasis | Giardia lamblia |
| Glanders | Burkholderia mallei |
| Gnathostomiasis | Gnathostoma spinigerum and Gnathostoma hispidum |
| Gonorrhea | Neisseria gonorrhoeae |
| Granuloma inguinale (Donovanosis) | Klebsiella granulomatis |
| Group A streptococcal infection | Streptococcus pyogenes |
| Group B streptococcal infection | Streptococcus agalactiae |
| Haemophilus influenzae infection | Haemophilus influenzae |
| Hand, foot and mouth disease (HFMD) | Enteroviruses, mainly Coxsackie A virus and Enterovirus |
| | 71 (EV71) |
| Hantavirus Pulmonary Syndrome (HPS) | Sin Nombre virus |
| Heartland virus disease | Heartland virus |
| Helicobacter pylori infection | Helicobacter pylori |
| Hemolytic-uremic syndrome (HUS) | Escherichia coli O157:H7, O111 and O104:H4 |
| Hemorrhagic fever with renal syndrome | Bunyaviridae family |
| (HFRS) | |
| Hepatitis A | Hepatitis A virus |
| Hepatitis B | Hepatitis B virus |
| Hepatitis C | Hepatitis C virus |
| Hepatitis D | Hepatitis D Virus |
| Hepatitis E | Hepatitis E virus |
| Herpes simplex | Herpes simplex virus 1 and 2 (HSV-1 and HSV-2) |
| Histoplasmosis | Histoplasma capsulatum |
| Hookworm infection | Ancylostoma duodenale and Necator americanus |
| Human bocavirus infection | Human bocavirus (HBoV) |
| Human ewingii ehrlichiosis | Ehrlichia ewingii |
| Human granulocytic anaplasmosis (HGA) | Anaplasma phagocytophilum |
| Human metapneumovirus infection | Human metapneumovirus (hMPV) |
| Human monocytic ehrlichiosis | Ehrlichia chaffeensis |
| Human papillomavirus (HPV) infection | Human papillomavirus (HPV) |
| Human parainfluenza virus infection | Human parainfluenza viruses (HPIV) |
| Hymenolepiasis | Hymenolepis nana and Hymenolepis diminuta |
| Epstein-Barr Virus Infectious | Epstein-Barr Virus (EBV) |
| Mononucleosis (Mono) | |
| Influenza (flu) | Orthomyxoviridae family |
| Isosporiasis | Isospora belli |
| Kawasaki disease | unknown; evidence supports that it is infectious |
| Keratitis | multiple |
| Kingella kingae infection | Kingella kingae |
| Kuru | PRNP |
| Lassa fever | Lassa virus |
| Legionellosis (Legionnaires' disease) | Legionella pneumophila |
| Legionellosis (Pontiac fever) | Legionella pneumophila |
| Leishmaniasis | Leishmania species |
| Leprosy | Mycobacterium leprae and Mycobacterium lepromatosis |
| Leptospirosis | Leptospira species |
| Listeriosis | Listeria monocytogenes |
| Lyme disease (Lyme borreliosis) | Borrelia burgdorferi, Borrelia garinii, and Borrelia afzelii |
| Lymphatic filariasis (Elephantiasis) | Wuchereria bancrofti and Brugia malayi |
| Lymphocytic choriomeningitis | Lymphocytic choriomeningitis virus (LCMV) |
| Malaria | Plasmodium species |
| Marburg hemorrhagic fever (MHF) | Marburg virus |
| Measles | Measles virus |
| Middle East respiratory syndrome (MERS) | Middle East respiratory syndrome coronavirus |
| Melioidosis (Whitmore's disease) | Burkholderia pseudomallei |
| Meningitis | multiple |
| Meningococcal disease | Neisseria meningitidis |
| Metagonimiasis | usually Metagonimus yokagawai |
| Microsporidiosis | Microsporidia phylum |
| Molluscum contagiosum (MC) | Molluscum contagiosum virus (MCV) |
| Monkeypox | Monkeypox virus |
| Mumps | Mumps virus |
| Murine typhus (Endemic typhus) | Rickettsia typhi |
| Mycoplasma pneumonia | Mycoplasma pneumoniae |
| Mycetoma (disambiguation) | numerous species of bacteria (Actinomycetoma) and |
| | fungi (Eumycetoma) |
| Myiasis | parasitic dipterous fly larvae |
| Neonatal conjunctivitis (Ophthalmia neonatorum) | most commonly Chlamydia trachomatis and Neisseria |
| | gonorrhoeae |
| (New) Variant Creutzfeldt-Jakob disease | PRNP |
| (vCJD, nvCJD) | |
| Nocardiosis | usually Nocardia asteroides and other Nocardia species |
| Onchocerciasis (River blindness) | Onchocerca volvulus |
| Opisthorchiasis | Opisthorchis viverrini and Opisthorchis felineus |
| Paracoccidioidomycosis (South American | Paracoccidioides brasiliensis |
| blastomycosis) | |
| Paragonimiasis | usually Paragonimus westermani and other Paragonimus |
| | species |
| Pasteurellosis | Pasteurella species |
| Pediculosis capitis (Head lice) | Pediculus humanus capitis |
| Pediculosis corporis (Body lice) | Pediculus humanus corporis |
| Pediculosis pubis (Pubic lice, Crab lice) | Phthirus pubis |
| Pelvic inflammatory disease (PID) | multiple |
| Pertussis (Whooping cough) | Bordetella pertussis |
| Plague | Yersinia pestis |
| Pneumococcal infection | Streptococcus pneumoniae |
| Pneumocystis pneumonia (PCP) | Pneumocystis jirovecii |
| Pneumonia | multiple |
| Poliomyelitis | Poliovirus |
| Prevotella infection | Prevotella species |
| Primary amoebic meningoencephalitis | usually Naegleria fowleri |
| (PAM) | |
| Progressive multifocal | JC virus |
| leukoencephalopathy | |
| Psittacosis | Chlamydophila psittaci |
| Q fever | Coxiella burnetii |
| Rabies | Rabies virus |
| Relapsing fever | Borrelia hermsii, Borrelia recurrentis, and other Borrelia |
| | species |
| Respiratory syncytial virus infection | Respiratory syncytial virus (RSV) |
| Rhinosporidiosis | Rhinosporidium seeberi |
| Rhinovirus infection | Rhinovirus |
| Rickettsial infection | Rickettsia species |
| Rickettsialpox | Rickettsia akari |
| Rift Valley fever (RVF) | Rift Valley fever virus |
| Rocky Mountain spotted fever (RMSF) | Rickettsia rickettsii |
| Rotavirus infection | Rotavirus |
| Rubella | Rubella virus |
| Salmonellosis | Salmonella species |
| SARS (Severe Acute Respiratory | SARS coronavirus |
| Syndrome) | |
| Scabies | Sarcoptes scabiei |
| Schistosomiasis | Schistosoma species |
| Sepsis | multiple |
| Shigellosis (Bacillary dysentery) | Shigella species |
| Shingles (Herpes zoster) | Varicella zoster virus (VZV) |
| Smallpox (Variola) | Variola major or Variola minor |
| Sporotrichosis | Sporothrix schenckii |
| Staphylococcal food poisoning | Staphylococcus species |
| Staphylococcal infection | Staphylococcus species |
| Strongyloidiasis | Strongyloides stercoralis |
| Subacute sclerosing panencephalitis | Measles virus |
| Syphilis | Treponema pallidum |
| Taeniasis | Taenia species |
| Tetanus (Lockjaw) | Clostridium tetani |
| Tinea barbae (Barber's itch) | usually Trichophyton species |
| Tinea capitis (Ringworm of the Scalp) | usually Trichophyton tonsurans |
| Tinea corporis (Ringworm of the Body) | usually Trichophyton species |
| Tinea cruris (Jock itch) | usually Epidermophyton floccosum, Trichophyton |
| | rubrum, and Trichophyton mentagrophytes |
| Tinea manum (Ringworm of the Hand) | Trichophyton rubrum |
| Tinea nigra | usually Hortaea werneckii |
| Tinea pedis (Athlete's foot) | usually Trichophyton species |
| Tinea unguium (Onychomycosis) | usually Trichophyton species |
| Tinea versicolor (Pityriasis versicolor) | Malassezia species |
| Toxocariasis (Ocular Larva Migrans | Toxocara canis or Toxocara cati |
| (OLM)) | |
| Toxocariasis (Visceral Larva Migrans | Toxocara canis or Toxocara cati |
| (VLM)) | |
| Trachoma | Chlamydia trachomatis |
| Toxoplasmosis | Toxoplasma gondii |
| Trichinosis | Trichinella spiralis |
| Trichomoniasis | Trichomonas vaginalis |
| Trichuriasis (Whipworm infection) | Trichuris trichiura |
| Tuberculosis | usually Mycobacterium tuberculosis |
| Tularemia | Francisella tularensis |
| Typhoid fever | Salmonella enterica subsp. enterica, serovar typhi |
| Typhus fever | Rickettsia |
| Ureaplasma urealyticum infection | Ureaplasma urealyticum |
| Valley fever | Coccidioides immitis or Coccidioides posadasii.[1] |
| Venezuelan equine encephalitis | Venezuelan equine encephalitis virus |
| Venezuelan hemorrhagic fever | Guanarito virus |
| Vibrio vulnificus infection | Vibrio vulnificus |
| Vibrio parahaemolyticus enteritis | Vibrio parahaemolyticus |
| Viral pneumonia | multiple viruses |
| West Nile Fever | West Nile virus |
| White piedra (Tinea blanca) | Trichosporon beigelii |
| Yersinia pseudotuberculosis infection | Yersinia pseudotuberculosis |
| Yersiniosis | Yersinia enterocolitica |
| Yellow fever | Yellow fever virus |

### Dose and Administration

The compositions, as described herein, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result.

Generally, the dose of the anti-chemorepellant agent of the present invention is from about 0.001 to about 100 mg/kg body weight per day, e.g., about 5 mg/kg body weight per day to about 50 mg/kg per day, e.g., about 5, 10, 15, 20, 25, 30, 35, 40, 45, or 50 mg/kg per day inclusive of all values and ranges therebetween, including endpoints. In one embodiment, the dose is from about 10 mg/kg to about 50 mg/kg per day. In one embodiment, the dose is from about 10 mg/kg to about 40 mg/kg per day. In one embodiment, the dose is from about 10 mg/kg to about 30 mg/kg per day. In one embodiment, the dose is from about 10 mg/kg to about 20 mg/kg per day. In one embodiment, the dose does not exceed about 50 mg/kg per day.

In one embodiment, the dose of the anti-chemorepellant agent is from about 50 mg/kg per week to about 350 mg/kg per week, inclusive of all values and ranges therebetween, including endpoints. In one embodiment, the dose of the anti-chemorepellant agent is about 50 mg/kg per week. In one embodiment, the dose of the anti-chemorepellant agent is about 60 mg/kg per week. In one embodiment, the dose of the anti-chemorepellant agent is about 70 mg/kg per week. In one embodiment, the dose of the anti-chemorepellant agent is about 80 mg/kg per week. In one embodiment, the dose of the anti-chemorepellant agent is about 90 mg/kg per week. In one embodiment, the dose of the anti-chemorepellant agent is about 100 mg/kg per week. In one embodiment, the dose of the anti-chemorepellant agent is about 110 mg/kg per week. In one embodiment, the dose of the anti-chemorepellant agent is about 120 mg/kg per week. In one embodiment, the dose of the anti-chemorepellant agent is about 130 mg/kg per week. In one embodiment, the dose of the anti-chemorepellant agent is about 140 mg/kg per week. In one embodiment, the dose of the anti-chemorepellant agent is about 150 mg/kg per week. In one embodiment, the dose of the anti-chemorepellant agent is about 160 mg/kg per week. In one embodiment, the dose of the anti-chemorepellant agent is about 170 mg/kg per week. In one embodiment, the dose of the anti-chemorepellant agent is about 180 mg/kg per week. In one embodiment, the dose of the anti-chemorepellant agent is about 190 mg/kg per week. In one embodiment, the dose of the anti-chemorepellant agent is about 200 mg/kg per week. In one embodiment, the dose of the anti-chemorepellant agent is about 210 mg/kg per week. In one embodiment, the dose of the anti-chemorepellant agent is about 220 mg/kg per week. In one embodiment, the dose of the anti-chemorepellant agent is about 230 mg/kg per week. In one embodiment, the dose of the anti-chemorepellant agent is about 240 mg/kg per week. In one embodiment, the dose of the anti-chemorepellant agent is about 250 mg/kg per week. In one embodiment, the dose of the anti-chemorepellant agent is about 260 mg/kg per week. In one embodiment, the dose of the anti-chemorepellant agent is about 270 mg/kg per week. In one embodiment, the dose of the anti-chemorepellant agent is about 280 mg/kg per week. In one embodiment, the dose of the anti-chemorepellant agent is about 290 mg/kg per week. In one embodiment, the dose of the anti-chemorepellant agent is about 300 mg/kg per week. In one embodiment, the dose of the anti-chemorepellant agent is about 310 mg/kg per week. In one embodiment, the dose of the anti-chemorepellant agent is about 320 mg/kg per week. In one embodiment, the dose of the anti-chemorepellant agent is about 330 mg/kg per week. In one embodiment, the dose of the anti-chemorepellant agent is about 340 mg/kg per week. In one embodiment, the dose of the anti-chemorepellant agent is about 350 mg/kg per week.

In one aspect of the invention, administration of the anti-chemorepellant agent is pulsatile. In one embodiment, an amount of anti-chemorepellant agent is administered every 1 hour to every 24 hours, for example every 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours. In one embodiment, an amount of anti-chemorepellant agent is administered every 1 day, 2 days, 3 days, 4 days, 5 days, 6 days, 7 days, 8 days, 9 days, or 10 days.

In one aspect of the invention, doses of the anti-chemorepellant agent are administered in a pulsatile manner for a period of time sufficient to have an anti-chemorepellant effect (e.g., to attenuate the chemorepellant effect of the pathogen). In one embodiment, the period of time is between about 1 day and about 10 days. For example, the period of time may be 1 day, 2 days, 3 days, 4 days, 5 days, 6 days, 7 days, 8 days, 9 days, or 10 days. In some embodiments, the APCs or T cells are administered after the anti-chemorepellant effect has occurred, e.g., one or more days after the anti-chemorepellant agent has been administered. In some embodiments, administration of the chemorepellant agent is continued while the APCs or T cells are being administered.

A variety of administration routes are available. The methods of the invention, generally speaking, may be practiced using any mode of administration that is medically acceptable, meaning any mode that produces effective levels of the active compounds without causing clinically unacceptable adverse effects.

Modes of administration include oral, rectal, topical, nasal, intradermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule. When peptides are used therapeutically, in certain embodiments a desirable route of administration is by pulmonary aerosol. Techniques for preparing aerosol delivery systems containing peptides are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody or peptide aerosols without resort to undue experimentation.

Compositions suitable for oral administration may be presented as discrete units, such as capsules, tablets, lozenges, each containing a predetermined amount of the active agent(s). Other compositions include suspensions in aqueous liquids or non-aqueous liquids such as a syrup, elixir or an emulsion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

In one embodiment, the anti-chemorepellant agent is administered parenterally. In one embodiment, the anti-chemorepellant agent is administered via microcatheter into a blood vessel proximal to pathogen or pathogen-infected cells. In one embodiment, the anti-chemorepellant agent is administered via microcatheter into a blood vessel within the tissue infected by pathogen. In one embodiment, the anti-chemorepellant agent is administered subcutaneously. In one embodiment, the anti-chemorepellant agent is administered intradermally.

Other delivery systems can include time-release, delayed release or sustained release delivery systems. Such systems can avoid repeated administrations of the anti-chemorepellant agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; sylastic systems; peptide based systems; wax coatings; compressed tablets using conventional binders and excipients; partially fused implants; and the like.

In one embodiment, the anti-chemorepellant agent or the APCs (e.g., dendritic cells) or activated T cells is administered in a time-release, delayed release or sustained release delivery system. In one embodiment, the time-release, delayed release or sustained release delivery system comprising the anti-chemorepellant agent or the APCs (e.g., dendritic cells) or activated T cells is inserted directly into the pathogen-infected cells. In one embodiment, the time-release, delayed release or sustained release delivery system comprising the anti-chemorepellant agent or the APCs (e.g., dendritic cells) or activated T cells is implanted in the patient proximal to the pathogen-infected cells. Additional implantable formulations are described, for example, in U.S. Patent App. Pub. No. 2008/0300165, which is incorporated herein by reference in its entirety.

In addition, important embodiments of the invention include pump-based hardware delivery systems, some of which are adapted for implantation. Such implantable pumps include controlled-release microchips. An exemplary controlled-release microchip is described in Santini, J T Jr. et al., Nature, 1999, 397:335-338, the contents of which are expressly incorporated herein by reference.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Dosages, methods, and means of administration of anti-pathogen agents are well-known in the art and can be determined by a skilled clinician.

### Pharmaceutical Compositions

In one aspect, this invention relates to a pharmaceutical composition comprising an anti-chemorepellant agent and APCs (e.g., dendritic cells) or activated T cells as described herein. In one embodiment, the APCs (e.g., dendritic cells) or T cells are derived from the patient to be treated. In one embodiment, the APCs (e.g., dendritic cells) or T cells express CXCR4 and/or CXCR7 on the cell surface.

In one embodiment, the composition further comprises a fusion protein as described herein.

In one embodiment, the composition comprises an effective amount of the fusion protein to activate the antigen-presenting cells. In one embodiment, the composition comprises an effective amount of the anti-chemorepellant agent to reduce the chemorepellant effect of the pathogen. In one embodiment, the composition comprises an effective amount of the antigen presenting cells to result in activation of T cells against the tumor.

In one embodiment, the composition further comprises an anti-pathogen agent.

In one embodiment, the pharmaceutical composition is formulated for injection.

In one embodiment, the composition comprises a pharmaceutically acceptable excipient. Acceptable excipients are non-toxic, aid administration, and do not adversely affect the therapeutic benefit of the claimed compounds. Such excipient may be any solid, liquid, semi-solid or gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols. Other suitable pharmaceutical excipients and their formulations are described in Remington's Pharmaceutical Sciences, edited by E. W. Martin (Mack Publishing Company, 22nd ed., 2013).

In order to ensure that the APCs or T cells and fusion protein interact once administered to a patient, the cells and protein may be combined prior to administration. In one embodiment, the cells and protein are combined immediately (e.g., seconds to one or two hours) prior to administration to the patient.

Without being bound by theory, it is believed that combination of the APCs or T cells and the anti-chemorepellant agent prior to administration to the patient will allow binding of the anti-chemorepellant agent to cell surface receptors (e.g., CXCR4 and/or CXCR7), thereby allowing the cells to overcome the chemorepellant effect of the pathogen. In contrast, it is contemplated that the systemic delivery of an anti-chemorepellant agent results in indiscriminate binding of that agent to CXCR4 receptors and/or CXCR7 throughout the body.

In one embodiment, this invention relates to an ex vivo device or container comprising activated APCs and T cells as described herein. The device or container is preferably capable of being maintained under controlled, reproducible conditions such that the activated APCs and T cells can interact to activate the T cells. For example, the device or container may be, without limitation, a cell culture dish or plate, a bag (e.g., a culture bag), or any other device or container that is suitable for growing and/or maintaining cells; a tube or column; a bioreactor; etc.

### Kit of Parts

In one aspect, this invention relates to a kit of parts for treatment of an infectious disease, the kit comprising APCs and/or activated T cells. In one embodiment, the kit further comprises an anti-pathogen agent, a fusion protein, and/or an anti-chemorepellant agent.

In one embodiment is provided a kit of parts for treatment of an infectious disease or a pathogen in a patient, the kit comprising a therapeutically effective amount of an anti-chemorepellant agent and the APCs (e.g., dendritic cells) and/or activated T cells prepared by the methods described herein.

In one embodiment, the anti-chemorepellant agent is selected from the group consisting of AMD3100 or a derivative thereof, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, and an antibody that interferes with dimerization of a receptor for a chemorepellant chemokine. In one embodiment, the anti-chemorepellant agent is AMD3100 or a derivative thereof. In one embodiment, the anti-chemorepellant agent is AMD3100.

In one embodiment, the APCs and/or activated T cells and/or the anti-chemorepellant agent are formulated for injection.

In one embodiment, the APCs and/or activated T cells and the anti-chemorepellant agent are in the same formulation. In one embodiment, the anti-pathogen agent is in the same formulation as the APCs and/or T cells and/or the anti-chemorepellant agent. In one embodiment, one or more of the anti-chemorepellant agent, APCs and/or T cells, and/or anti-pathogen agent are in separate formulation(s).

In one embodiment, the kit further comprises instructions for treating an infectious disease. In one embodiment, the kit of parts comprises instructions in a readable medium for dosing and/or administration of the anti-chemorepellant agent and the APCs and/or activated T cells.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (“ROM”); random access memory (“RAM”); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (“QR Code”) or other matrix barcode.

### EXAMPLES

### Example 1. Fusion Protein Targeting HBV

Rationale:

Current antiviral treatment of chronic hepatitis B (HBV) results in sustainable host viral control in only 25% of infected individuals. Alternative therapeutic vaccine approaches focused on boosting T cell responses in these patients to achieve viral clearance have proven challenging due to the exhausted and suppressed state of HBV-specific T cells. While a dendritic cell vaccine approach shows promise in reversing T cell exhaustion or suppression, clinical use of such vaccines will be individualized, complex and expensive. In this context an alternative approach that uses a scalable recombinant protein to induce the same type of effects as dendritic cell vaccines would offer a solution more amenable for wide application throughout the military medical system. Such an approach could be built on the use of Mycobacterium tuberculosis heat shock protein 70 (MtbHSP70), which can target dendritic cells, promote both uptake and class I and class II processing of pathogen antigens, and stimulate release of immunostimulatory cytokines that activate and enhance maturation of these dendritic cells. These advantages to using MtbHSP70 with hepatitis B vaccines have been demonstrated in preclinical vaccines that incorporate specific hepatitis B antigens or epitopes for standard vaccination. The proposed approach fuses MtbHSP70 to a single chain variable fragment (scFv) capable of high-affinity binding to a HBV surface antigen (HBsAg). The fusion protein binds to virus via the scFv, while the MtbHSP70 portion both stimulates dendritic cells and induces virus uptake, resulting in cross-presentation of viral antigens to T cells and B cells by activated dendritic cells. The fusion protein effectively induces an in situ immune response in both the blood and liver, where virus may be circulating or replicating, respectively.

Hypothesis:

A scFv-MtbHSP70 fusion protein targeting HBsAg on circulating/liver HBV virions can induce uptake and cross-presentation of multiple HBV antigens by dendritic cells leading to significant multivalent, viral-specific T cell and B cell responses in a transgenic mouse model of chronic HBV infection.

Innovation:

The central innovation of this project is to induce the immunologic responses found in dendritic cell vaccination by using a scalable therapeutic protein that can facilitate “in vivo dendritic cell vaccination.” This novel immunotherapy approach for overcoming immune tolerance or exhaustion in HBV infection could contribute to strategies for eliciting sustained immunological control of chronic HBV, either alone or in combination with standard of care antiviral therapies, and offer hope of an effective curative therapy.

Study Design:

A scFv-MtbHSP70 fusion protein targeting HBsAg is constructed and characterized for research use. Initial in vitro studies demonstrate functional integrity of both the scFv (binding affinity to HBsAg) and MtbHSP70 (ATPase activity) components. The immune effects of the therapeutic protein are tested in HBV transgenic mice. Mice treated with the fusion protein or with MtbHSP70 alone and PBS as negative controls are evaluated for cellular (IFNγ production by antigen-stimulated splenocytes) and humoral (serum IgG) responses to both HBSAg antigen and HBV core protein. Liver histology, liver HBV DNA content, and serum alanine aminotransferase levels are assessed as indicators of antiviral cytotoxic responses. In addition, the immune system's response to the targeted immunotherapy is characterized using cytometry by time-of-flight mass spectrometry (CyTOF) for simultaneous measurement of >35 immune parameters.

Expected Results:

Results from this project demonstrating activation of immune responses to HBV in the transgenic mouse model will be used to develop a preclinical therapeutic candidate for clinical evaluation in chronic HBV patients.

The foregoing is illustrative of the present invention, and is not to be construed as limiting thereof. The invention is defined by the following claims, with equivalents of the claims to be included therein.

1. A method for preparing activated antigen-presenting cells (APCs), the method comprising:
(a) incubating immune cells ex vivo in the presence of a pathogen sample and a fusion protein for a time period sufficient to produce activated APCs, wherein at least one activated APC displays an antigen derived from the pathogen sample; and
(b) isolating the activated APCs;
wherein the fusion protein comprises an antigen binding component and a stress protein component, wherein said antigen binding component binds to an antigen from the pathogen sample, and said stress protein component activates the APCs.

2-25. (canceled)

26. A method for treating a disease caused by a pathogen in a patient, the method comprising administering an effective amount of ex vivo prepared activated APCs to the patient;
wherein the APCs were prepared by the method of claim 1.

27-70. (canceled)

71. An ex vivo composition comprising activated APCs and an effective amount of a fusion protein, an anti-chemorepellant agent, and/or an anti-pathogen agent.

72. (canceled)

73. The composition of claim 71, wherein the fusion protein comprises a pathogen binding component and a stress protein component.

74. The composition of claim 71, wherein the activated APCs comprise dendritic cells, B lymphocytes, and/or mononuclear phagocytes.

75. The composition of claim 71, wherein at least one activated APC expresses a CD40 receptor.

76. The composition of claim 71, wherein the activated APCs comprise activated dendritic cells.

77. The composition of claim 71, further comprising a growth factor.

78. The composition of claim 77, wherein the growth factor comprises a cytokine.

79. The composition of claim 77, wherein the growth factor is selected from a group consisting of Flt-3 ligand, GM-CSF, IL-4, M-CSF, IFNα, IL-1β, IL-4, IL-6, IL-13, IL-15 and TNFα.

80. A pharmaceutical composition comprising the composition of claim 71.

81. The composition of claim 80, further comprising an effective amount of a fusion protein, an anti-chemorepellant agent, and/or an anti-pathogen agent.

82. The composition of claim 81, wherein the anti-chemorepellant agent is selected from the group consisting of AMD3100 or a derivative thereof, AMD11070, AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, TN14003, TAK-779, AK602, SCH-351125, tannic acid, NSC 651016, thalidomide, GF 109230X, an antibody that interferes with dimerization of a chemorepellant chemokine, and an antibody that interferes with dimerization of a receptor for a chemorepellant chemokine.

83. The composition of claim 82, wherein the anti-fugetactic agent is AMD3100.

84. The composition of claim 81, wherein the fusion protein comprises a stress protein domain and an antigen-binding domain, wherein the stress protein domain activates APCs and the antigen-binding domain is capable of binding an antigen derived from the pathogen or from cells infected with the pathogen.

85. The composition of claim 81, wherein the anti-pathogen agent comprises an antimicrobial agent, an antibiotic, and anti-fungal agent, an anti-protozoa agent, or an anti-parasitic agent.

86. The composition of claim 80, wherein the activated APCs comprise activated dendritic cells.

87-88. (canceled)

89. A method for preparing activated T cells, the method comprising:
a) providing activated antigen-presenting cells (APCs) that were activated by the method of claim 1, wherein at least one activated antigen-presenting cell displays an antigen derived from the pathogen sample;
b) contacting the activated antigen-presenting cells with T cells for a period of time sufficient to activate the T cells; and
c) isolating the activated T cells.

90-108. (canceled)

109. A method for treating a pathogen infection in a patient, the method comprising administering an effective amount of activated T cells to the patient, wherein the activated T cells were prepared by the method of claim 89.

110-140. (canceled)

141. A pharmaceutical composition comprising activated T cells and an anti-chemorepellant agent.

142-146. (canceled)
